(19) United States
(12) Patent Application Publication
(10) Pub. No.: US 2026/0251631 A1
(43) Pub. Date: Aug. 27, 2026

(54) ASSESSING AND MANAGING THE IMPACT OF SUBSURFACE SCALE FORMATION ON HYDROCARBON RECOVERY

(71) Applicant: Chevron U.S.A. Inc., San Ramon, CA (US)

(72) Inventors: Wei Wei, Sugar Land, TX (US); Hyuntae Yang, Houston, TX (US); Wei Wang, Houston, TX (US); Gregory Arthur Winslow, Houston, TX (US); Guo-Qing Tang, Mountain View, CA (US); Haiping Lu, Sugar Land, TX (US)

(21) Appl. No.: 19/545,703

(22) Filed: Feb. 20, 2026

Related U.S. Application Data
(60) Provisional application No. 63/761,794, filed on Feb. 21, 2025.

(51) Int. Cl. G01N 33/24; E21B 41/02

(52) U.S. Cl. CPC G01N 33/24; E21B 41/02

(57) ABSTRACT

A method for assessing impacts of subsurface scale formation from fluid flow to improve hydrocarbon recovery from a fractured subterranean formation may include obtaining first measurements of a parameter associated with a first interaction between a first core sample and a first fluid, where the first fluid includes a first brine without a scale inducer. The method also includes establishing a baseline of the parameter using the first measurements. The method further includes obtaining second measurements of the parameter associated with a second interaction between a second core sample and a second fluid, and where the second fluid includes a second brine and a scale impact additive. The method also includes comparing the second measurements to the baseline and identifying a target fluid based on the comparison.

### CROSS-REFERENCE TO RELATED APPLICATIONS

This application claims priority to U.S. Provisional Patent Application Ser. No. 63/761,794 titled “Assessing and Managing the Impact Of Subsurface Scale Formation On Hydrocarbon Recovery” and filed on Feb. 21, 2025, the entire contents of which are hereby incorporated by reference.

### TECHNICAL FIELD

The present application is related to subterranean field operations and, more particularly, to assessing and managing the impact of subsurface scale formation on hydrocarbon recovery.

### BACKGROUND

Some subterranean formations, such as shale, may produce subterranean resources through techniques such as horizontal drilling and fracturing. Over time, the fractures may become restricted or blocked. Preventing or reducing the development and growth of these restrictions or blockages may lead to enhanced extraction of the subterranean resources for an extended period of time. In some cases, such as with unconventional formations, subsurface scale formation may lead to underperformance in well production and oil recovery.

### SUMMARY

In general, in one aspect, the disclosure relates to a method for assessing impacts of subsurface scale formation from fluid flow to improve hydrocarbon recovery from a fractured subterranean formation. The method may include obtaining a plurality of first measurements, made by a sensor device, of a parameter associated with a first interaction between a first core sample and a first fluid of a plurality of fluids inside of a testing vessel, where the first core sample is extracted from a depth range of a wellbore in the fractured subterranean formation, and where the first fluid comprises a first brine without a scale inducer. The method may also include establishing a baseline of the parameter using the plurality of first measurements. The method may further include obtaining a plurality of second measurements, made by the sensor device, of the parameter associated with a second interaction between a second core sample and a second fluid of the plurality of fluids inside of the testing vessel, where the second core sample is extracted from the depth range of the wellbore in the fractured subterranean formation, and where the second fluid comprises a second brine and a scale impact additive. The method may also include comparing the plurality of second measurements to the baseline. The method may further include identifying, based on comparing the plurality of second measurements to the baseline, a target fluid for use in production of a hydrocarbon from the fractured subterranean formation within the depth range.

In another aspect, the disclosure relates to system for assessing impacts of subsurface scale formation from fluid flow to improve hydrocarbon recovery from a fractured subterranean formation. The system may include a testing module comprising a testing vessel, wherein the testing vessel is configured to receive a plurality of core samples extracted from a depth range of a wellbore in the fractured subterranean formation, where the testing module is configured to control a pressure and a temperature of the testing vessel. The testing module may also be configured to facilitate a first fluid of a plurality of fluids that interacts with a first core sample of the plurality of core samples in the testing vessel for a first period of time, where the first fluid includes a first brine without a scale inducer. The testing module may further be configured to facilitate a second fluid of the plurality of fluids that interacts with a second core sample of the plurality of core samples in the testing vessel for a second period of time, where the second fluid comprises a second brine and a scaling ion.

These and other aspects, objects, features, and embodiments will be apparent from the following description and the appended claims.

### BRIEF DESCRIPTION OF THE DRAWINGS

The drawings illustrate only example embodiments and are therefore not to be considered limiting in scope, as the example embodiments may admit to other equally effective embodiments. The elements and features shown in the drawings are not necessarily to scale, emphasis instead being placed upon clearly illustrating the principles of the example embodiments. Additionally, certain dimensions or positions may be exaggerated to help visually convey such principles. In the drawings, reference numerals designate like or corresponding, but not necessarily identical, elements.

FIGS. 1A through 1C show a field system, and details thereof, with which example embodiments may be used.

FIG. 2 shows the detail of a fracture of the field system of FIG. 1A during drilling or completion of the wellbore for which example embodiments may be used.

FIG. 3 shows the detail of a fracture of the field system of FIG. 1A during a shut-in period of the wellbore for which example embodiments may be used.

FIG. 4 shows the detail of FIG. 1C at a subsequent point in time according to certain example embodiments.

FIG. 5 shows the detail of FIG. 4 at a subsequent point in time according to certain example embodiments.

FIG. 6 shows a diagram of a testing system for assessing impacts of subsurface scale formation to improve hydrocarbon recovery from a fractured subterranean formation according to certain example embodiments.

FIG. 7 shows a system diagram of a controller of FIG. 6 according to certain example embodiments.

FIG. 8 shows a computing device in accordance with certain example embodiments.

FIG. 9 shows a flowchart of a method for assessing impacts of subsurface scale formation to improve hydrocarbon recovery from a fractured subterranean formation according to certain example embodiments.

FIGS. 10 through 18 show graphs based on results of a series of experiments conducted on core samples according to certain example embodiments.

FIG. 19 shows a graph of oil production rates for multiple wellbores over time according to certain example embodiments.

FIG. 20 shows an image of a testing system that is modeled after the testing system of FIG. 6 according to certain example embodiments.

### DETAILED DESCRIPTION

The example embodiments discussed herein are directed to systems, apparatus, methods, and devices for assessing impacts of subsurface scale formation to improve hydrocarbon recovery from a fractured subterranean formation. An assessment of impacts of subsurface scale formation may result in reducing subsurface scale formation (e.g., reducing deposition of scales and/or other solids), which may involve any of a number of different actions. For example, reducing deposition of scales and/or other solids may include minimizing the accumulation or deposition of scales and/or other solids without completely eliminating the scales and/or other solids. As another example, reducing deposition of scales and/or other solids as defined herein may additionally or alternatively mean preventing the development of scales and/or other solids. As yet another example, reducing deposition of scales and/or other solids as defined herein may additionally or alternatively mean completely eliminating scales and/or other solids that have previously developed.

The use of the terms “about”, “approximately”, and similar terms applies to all numeric values, whether or not explicitly indicated. These terms generally refer to a range of numbers that one of ordinary skill in the art would consider as a reasonable amount of deviation to the recited numeric values (i.e., having the equivalent function or result). For example, this term may be construed as including a deviation of ±10 percent of the given numeric value provided such a deviation does not alter the end function or result of the value. Therefore, a value of about 1% may be construed to be a range from 0.9% to 1.1%. Furthermore, a range may be construed to include the start and the end of the range. For example, a range of 10% to 20% (i.e., range of 10%-20%) includes 10% and also includes 20%, and includes percentages in between 10% and 20%, unless explicitly stated otherwise herein. Similarly, a range of between 10% and 20% (i.e., range between 10%-20%) includes 10% and also includes 20%, and includes percentages in between 10% and 20%, unless explicitly stated otherwise herein. Use of the term “configured to” herein is equivalent to the term “designed to” or “specifically designed to” or “structured and arranged to”, as opposed to a broader term such as “capable of”.

A “subterranean formation” refers to practically any volume under a surface. For example, it may be practically any volume under a terrestrial surface (e.g., a land surface), practically any volume under a seafloor, etc. Each subsurface volume of interest may have a variety of characteristics, such as petrophysical rock properties, reservoir fluid properties, reservoir conditions, hydrocarbon properties, or any combination thereof. For example, each subsurface volume of interest may be associated with one or more of: temperature, porosity, salinity, permeability, water composition, mineralogy, hydrocarbon type, hydrocarbon quantity, reservoir location, pressure, etc. Those of ordinary skill in the art will appreciate that the characteristics are many, including, but not limited to: shale gas, shale oil, tight gas, tight oil, tight carbonate, carbonate, vuggy carbonate, unconventional (e.g., a permeability of less than 25 millidarcy (mD) such as a permeability of from 0.000001 mD to 25 mD)), diatomite, geothermal, mineral, etc. The terms “formation”, “subsurface formation”, “hydrocarbon-bearing formation”, “reservoir”, “subsurface reservoir”, “subsurface area of interest”, “subsurface region of interest”, “subsurface volume of interest”, and the like may be used synonymously. The term “subterranean formation” is not limited to any description or configuration described herein.

A “well” or a “wellbore” refers to a single hole, usually cylindrical, that is drilled into a subsurface volume of interest. A well or a wellbore may be drilled in one or more directions. For example, a well or a wellbore may include a vertical well, a horizontal well, a deviated well, and/or other type of well. A well or a wellbore may be drilled in the subterranean formation for exploration and/or recovery of resources. A plurality of wells (e.g., tens to hundreds of wells) or a plurality of wellbores are often used in a field depending on the desired outcome.

A well or a wellbore may be drilled into a subsurface volume of interest using practically any drilling technique and equipment known in the art, such as geosteering, directional drilling, etc. Drilling the well may include using a tool, such as a drilling tool that includes a drill bit and a drill string. Drilling fluid, such as drilling mud, may be used while drilling in order to cool the drill tool and remove cuttings. Other tools may also be used while drilling or after drilling, such as measurement-while-drilling (MWD) tools, seismic-while-drilling (SWD) tools, wireline tools, logging-while-drilling (LWD) tools, or other downhole tools. After drilling to a predetermined depth, the drill string and the drill bit may be removed, and then the casing, the tubing, and/or other equipment may be installed according to the design of the well. The equipment to be used in drilling the well may be dependent on the design of the well, the subterranean formation, the hydrocarbons, and/or other factors.

A well may include a plurality of components, such as, but not limited to, a casing, a liner, a tubing string, a sensor, a packer, a screen, a gravel pack, artificial lift equipment (e.g., an electric submersible pump (ESP)), and/or other components. If a well is drilled offshore, the well may include one or more of the previous components plus other offshore components, such as a riser. A well may also include equipment to control fluid flow into the well, control fluid flow out of the well, or any combination thereof. For example, a well may include a wellhead, a choke, a valve, and/or other control devices. These control devices may be located on the surface, in the subsurface (e.g., downhole in the well), or any combination thereof. In some embodiments, the same control devices may be used to control fluid flow into and out of the well. In some embodiments, different control devices may be used to control fluid flow into and out of a well. In some embodiments, the rate of flow of fluids through the well may depend on the fluid handling capacities of the surface facility that is in fluidic communication with the well. The equipment to be used in controlling fluid flow into and out of a well may be dependent on the well, the subsurface region, the surface facility, and/or other factors. Moreover, sand control equipment and/or sand monitoring equipment may also be installed (e.g., downhole and/or on the surface). A well may also include any completion hardware that is not discussed separately. The term “well” may be used synonymously with the terms “borehole,” “wellbore,” or “well bore.” The term “well” is not limited to any description or configuration described herein.

Example embodiments of assessing impacts of subsurface scale formation to improve hydrocarbon recovery from a fractured subterranean formation may be at a subsurface (e.g., propped fractures, frac face, in or near perforations, within and adjacent to a wellbore in a subterranean formation). Example embodiments of assessing impacts of subsurface scale formation to improve hydrocarbon recovery from a fractured subterranean formation may additionally or alternatively be used in any of a number of other applications. For instance, example embodiments may be used to control the deposition of scales and/or other solids in production facilities. Such production facilities may include, but are not limited to, production tubing, heat exchangers, and conduit or other pipes (e.g., a pipeline) used to transport fluid (e.g., produced fluids from oil and gas wells).

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein. By way of example, if an item is described herein as including a component of type A, a component of type B, a component of type C, or any combination thereof, it is understood that this phrase describes all of the various individual and collective combinations and permutations of these components.

For example, in some embodiments, the item described as including a component of type A, a component of type B, a component of type C, or any combination thereof, it is understood that this phrase could include only a component of type A. In some embodiments, the item described as including a component of type A, a component of type B, a component of type C, or any combination thereof, it is understood that this phrase could include only a component of type B. In some embodiments, the item described as including a component of type A, a component of type B, a component of type C, or any combination thereof, it is understood that this phrase could include only a component of type C. In some embodiments, the item described as including a component of type A, a component of type B, a component of type C, or any combination thereof, it is understood that this phrase could include a component of type A and a component of type B. In some embodiments, the item described as including a component of type A, a component of type B, a component of type C, or any combination thereof, it is understood that this phrase could include a component of type A and a component of type C. In some embodiments, the item described as including a component of type A, a component of type B, a component of type C, or any combination thereof, it is understood that this phrase could include a component of type B and a component of type C. In some embodiments, the item described as including a component of type A, a component of type B, a component of type C, or any combination thereof, it is understood that this phrase could include a component of type A, a component of type B, and a component of type C.

In some embodiments, the item described as including a component of type A, a component of type B, a component of type C, or any combination thereof, it is understood that this phrase could include two or more components of type A (e.g., A1 and A2). In some embodiments, the item described as including a component of type A, a component of type B, a component of type C, or any combination thereof, it is understood that this phrase could include two or more components of type B (e.g., B1 and B2). In some embodiments, the item described as including a component of type A, a component of type B, a component of type C, or any combination thereof, it is understood that this phrase could include two or more components of type C (e.g., C1 and C2).

In some embodiments, the item described as including a component of type A, a component of type B, a component of type C, or any combination thereof, it is understood that this phrase could include two or more of a first component (e.g., two or more components of type A (A1 and A2)), optionally one or more of a second component (e.g., optionally one or more components of type B), and optionally one or more of a third component (e.g., optionally one or more components of type C). In some embodiments, the item described as including a component of type A, a component of type B, a component of type C, or any combination thereof, it is understood that this phrase could include two or more of a first component (e.g., two or more components of type B (B1 and B2)), optionally one or more of a second component (e.g., optionally one or more components of type A), and optionally one or more of a third component (e.g., optionally one or more components of type C).

In some embodiments, the item described as including a component of type A, a component of type B, a component of type C, or any combination thereof, it is understood that this phrase could include two or more of a first component (e.g., two or more components of type C (C1 and C2)), optionally one or more of a second component (e.g., optionally one or more components of type A), and optionally one or more of a third component (e.g., optionally one or more components of type B).

If a component of a figure is described but not expressly shown or labeled in that figure, the label used for a corresponding component in another figure may be inferred to that component. Conversely, if a component in a figure is labeled but not described, the description for such component may be substantially the same as the description for the corresponding component in another figure. The numbering scheme for the various components in the figures herein is such that each component is a three-digit number or a four-digit number, and corresponding components in other figures have the identical last two digits. For any figure shown and described herein, one or more of the components may be omitted, added, repeated, and/or substituted. Accordingly, embodiments shown in a particular figure should not be considered limited to the specific arrangements of components shown in such figure.

Further, a statement that a particular embodiment (e.g., as shown in a figure herein) does not have a particular feature or component does not mean, unless expressly stated, that such embodiment is not capable of having such feature or component. For example, for purposes of present or future claims herein, a feature or component that is described as not being included in an example embodiment shown in one or more particular drawings is capable of being included in one or more claims that correspond to such one or more particular drawings herein.

Example embodiments of assessing impacts of subsurface scale formation to improve hydrocarbon recovery from a fractured subterranean formation will be described more fully hereinafter with reference to the accompanying drawings, in which example embodiments of assessing impacts of subsurface scale formation to improve hydrocarbon recovery from a fractured subterranean formation are shown. Assessing impacts of subsurface scale formation to improve hydrocarbon recovery from a fractured subterranean formation may, however, be embodied in many different forms and should not be construed as limited to the example embodiments set forth herein. Rather, these example embodiments are provided so that this disclosure will be thorough and complete, and will fully convey the scope of assessing impacts of subsurface scale formation to improve hydrocarbon recovery from a fractured subterranean formation to those of ordinary skill in the art. Like, but not necessarily the same, elements (also sometimes called components) in the various figures are denoted by like reference numerals for consistency.

Terms such as “first”, “second”, “outer”, “inner”, “top”, “bottom”, “above”, “below”, “distal”, “proximal”, “front,”, “rear,” “left,” “right,” “on”, and “within”, when present, are used merely to distinguish one component (or part of a component or state of a component) from another. This list of terms is not exclusive. Such terms are not meant to denote a preference or a particular orientation, and they are not meant to limit embodiments of assessing impacts of subsurface scale formation to improve hydrocarbon recovery from a fractured subterranean formation. In the following detailed description of the example embodiments, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

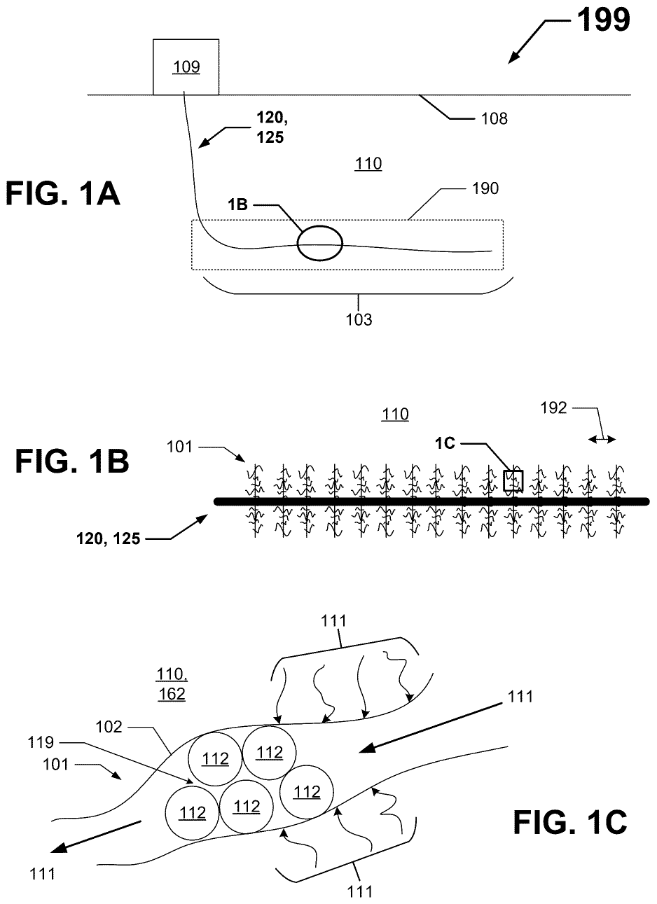

FIGS. 1A through 1C show a field system 199, including details thereof, with which example embodiments may be used. Specifically, FIG. 1A shows a schematic diagram of a land-based field system 199 in which a wellbore 120 has been drilled in a subterranean formation 110. FIG. 1B shows a detail of a substantially horizontal section 103 of the wellbore 120 of FIG. 1A. FIG. 1C shows a detail of a fracture 101 of FIG. 1B. The field system 199 in this example includes a wellbore 120 disposed in a subterranean formation 110 using field equipment 109 (e.g., a derrick, a tool pusher, a clamp, a tong, drill pipe, casing pipe, a drill bit, a wireline tool, a fluid pumping system) located above a surface 108 and within the wellbore 120. Once the wellbore 120 is drilled, a casing string 125 is inserted into the wellbore 120 to stabilize the wellbore 120 and allow for the extraction of subterranean resources (e.g., natural gas, oil) from the subterranean formation 110.

The surface 108 may be ground level for an onshore application and the sea floor/lakebed for an offshore application. For offshore applications, at least some of the field equipment may be located on a platform that sits above the water level. The point where the wellbore 120 begins at the surface 108 may be called the entry point and have a wellhead (e.g., an assembly of pipes, valves, and/or other equipment) positioned thereon. While not shown in FIGS. 1A and 1B, there may be multiple wellbores 120, each with its own wellhead but that is located close to the other wellheads, drilled into the subterranean formation 110 and having substantially horizontal sections 103 that are close to each other. In such a case, the multiple wellbores 120 may be drilled at the same pad or at different pads.

When the drilling process is complete, other operations, such as fracturing operations, may be performed. Fractures 101 in the subterranean formation 110 are shown to be located in the horizontal section 103 of the wellbore 120 in FIG. 1B. The fractures 101 may additionally or alternatively be located in other sections (e.g., a substantially vertical section, a transition area between a vertical section and a horizontal section 103) of the wellbore 120. Example embodiments may be used along any portion of the wellbore 120 where fractures 101 are located.

The subterranean formation 110 may include one or more of a number of formation types, including but not limited to shale, limestone, sandstone, clay, sand, and salt. In certain embodiments, a subterranean formation 110 may include one or more reservoirs in which one or more resources (e.g., oil, natural gas, water, steam) may be located. One or more of a number of field operations (e.g., fracturing, coring, tripping, drilling, setting casing, extracting downhole resources) may be performed to reach an objective of a user with respect to the subterranean formation 110.

The wellbore 120 may have one or more of a number of segments or hole sections, where each segment or hole section may have one or more of a number of dimensions. Examples of such dimensions may include, but are not limited to, a size (e.g., diameter) of the wellbore 120, a curvature of the wellbore 120, a total vertical depth of the wellbore 120, a measured depth of the wellbore 120, and a horizontal displacement of the wellbore 120. There may be multiple overlapping casing strings of various sizes (e.g., length, outer diameter) contained within and between these segments or hole sections to ensure the integrity of the wellbore construction. In this case, one or more of the segments of the subterranean wellbore 120 is the substantially horizontal section 103. As stated above, in additional or alternative cases, one or more of the segments of the subterranean wellbore 120 is a substantially vertical section.

As discussed above, inserted into and disposed within the wellbore 120 of FIGS. 1A and 1B are a number of casing pipes that are coupled to each other end-to-end to form the casing string 125. In this case, each end of a casing pipe has mating threads (a type of coupling feature) disposed thereon, allowing a casing pipe to be directly or indirectly mechanically coupled to another casing pipe in an end-to-end configuration. The casing pipes of the casing string 125 may be indirectly mechanically coupled to each other using a coupling device, such as a coupling sleeve.

Each casing pipe of the casing string 125 may have a length and a width (e.g., outer diameter). The length of a casing pipe may vary. For example, a common length of a casing pipe is approximately 40 feet. The length of a casing pipe may be longer (e.g., 60 feet) or shorter (e.g., 10 feet) than 40 feet. The width of a casing pipe may also vary and may depend on the cross-sectional shape of the casing pipe. For example, when the shape of the casing pipe is cylindrical, the width may refer to an outer diameter, an inner diameter, or some other form of measurement of the casing pipe. Examples of a width in terms of an outer diameter may include, but are not limited to, 4-½ inches, 7 inches, 7-⅝ inches, 8-⅝ inches, 10-¾ inches, 13-⅜ inches, and 14 inches.

The size (e.g., width, length) of the casing string 125 may be based on the information (e.g., diameter of the borehole drilled) gathered using field equipment with respect to the subterranean wellbore 120. The walls of the casing string 125 have an inner surface that forms a cavity that traverses the length of the casing string 125. Each casing pipe may be made of one or more of a number of suitable materials, including but not limited to steel. Cement is poured into the wellbore 120, often through the cavity and then forced upward between the outer surface of the casing string 125 and the wall of the subterranean wellbore 120. In some cases, a liner may additionally be used with, or alternatively be used in place of, some or all of the casing pipes.

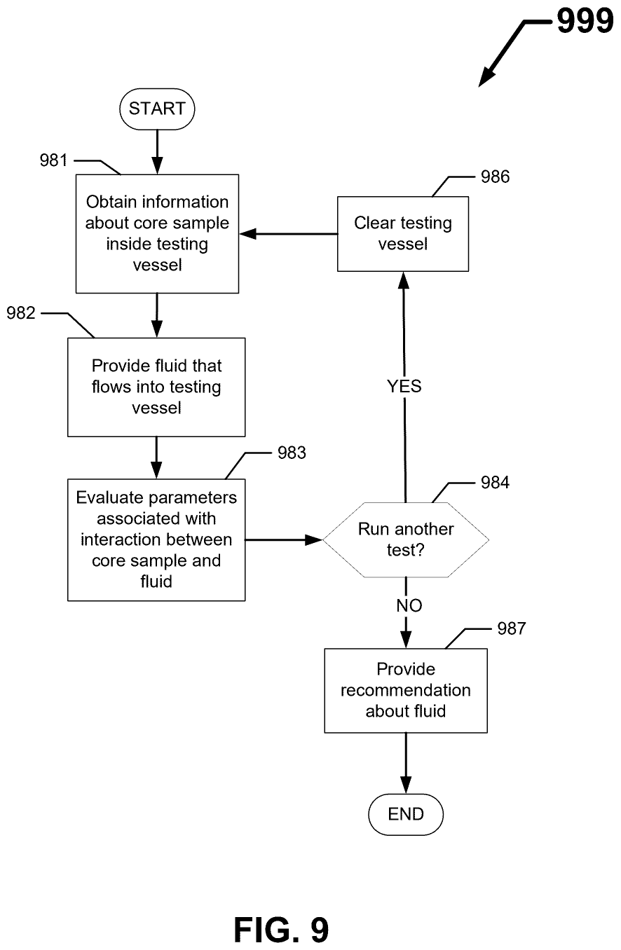

Once the cement dries, a number of fractures 101 are formed in the subterranean formation 110. The fractures 101 may be formed in any of a number of ways known in the industry, including but not limited to hydraulic fracturing and/or other methods. The hydraulic fracturing process involves the injection of large quantities of fluids (outside of the fluids discussed below with respect to FIG. 6) containing water, chemical additives, and proppants 112 (e.g., sand, ceramic pellets) into the subterranean formation 110 from the wellbore 120 to create fracture networks. A subterranean formation 110 naturally has fractures 101, but these naturally occurring fractures 101 have inconsistent characteristics (e.g., length, spacing) and so in some cases may not be relied upon for extracting subterranean resources without having additional fractures 101, such as what is shown in FIG. 1B, in the subterranean formation 110. When proppant 112 is used, some of the fractures 101 (also sometimes called principal or primary fractures) receive proppant 112, while a remainder of the fractures 101 (also sometimes called secondary fractures) do not have any proppant 112 in them.

As shown in FIG. 1C, the proppant 112 is designed to become lodged inside at least some of the fractures 101 to keep those fractures 101 open after the fracturing operation is complete. While the proppant 112 keeps a fracture 101 open, the proppant 112 also represents obstacles within the fracture 101 that restrict flow in the form of pore throats 119. In other words, the pore throats 119 are the passageways that result in the fracture 101 based on obstacles (e.g., proppant 112, grains) located in the fracture 101. The size of the proppant 112 is an important design consideration. Sizes (e.g., 40/70 mesh, 50/140 mesh) of the proppant 112 may vary. While the shape of the proppant 112 is shown as being uniformly spherical, and the size is substantially identical among the proppant 112, the actual sizes and/or shapes of the proppant 112 may vary. If the proppant 112 is too small, the proppant 112 will not be effective at keeping the fractures 101 open enough to effectively allow subterranean resources 111 to flow through the fractures 101 from the rock matrices 162 in the subterranean formation 110 to the wellbore 120. If the proppant 112 is too large, the proppant 112 may plug up the fractures 101 because the pore throats 119 become too small, blocking the flow of the subterranean resources 111 through the fractures 101.

The use of proppant 112 in certain types of subterranean formation 110, such as shale and other tight and/or unconventional formations, is important. Shale formations typically have permeabilities on the order of microdarcys (μD) to nanodarcys (nD). When fractures 101 are formed in such formations with low permeabilities, it is important to sustain the fractures 101 and their conductivity for an extended period of time in order to extract more of the subterranean resource 111.

The various fractures 101 that originate at the wellbore 120 and extend outward into the rock matrices 162 in the subterranean formation 110 in this case have consistent penetration lengths perpendicular to the wellbore 120 and have consistent coverage along at least a portion of the lateral length (substantially horizontal section 103) of the wellbore 120. For example, fractures 101 may be 50 meters high and 200 meters long. Further, the fractures 101 may be spaced a distance 192 apart from each other. The distance 192 (e.g., 25 meters, 5 meters, 12 meters) may be optimized based on characteristics such as the permeability and/or the porosity of the rock matrix 162 of the subterranean formation 110.

The fractures 101 create a volume 190 within the subterranean formation 110 where the rock matrix 162 of the subterranean formation 110 is connected to the high conductivity fractures 101 located a short distance away. In addition to different configurations of the fractures 101, other factors that may contribute to the viability of the subterranean formation 110 may include, but are not limited to, permeability of the rock matrix 162, capillary pressure, and the temperature and pressure of the subterranean formation 110. Each fracture 101 is defined by a wall, also called a frac face 102 herein. The frac face 102 provides a transition between the paths formed by the rock matrices 162 in the subterranean formation 110 and the fracture 101. The subterranean resources 111 flow through the paths formed by the rock matrices 162 in the subterranean formation 110 into the fracture 101.

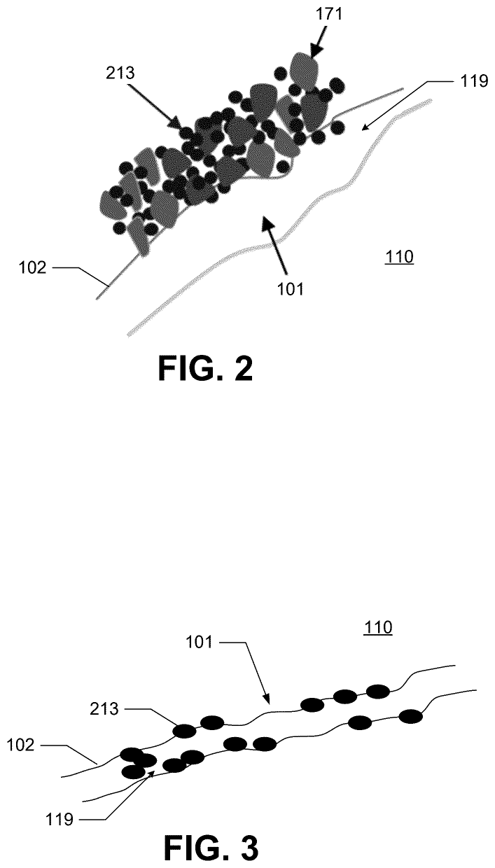

FIG. 2 shows the detail of a fracture 101 of the field system 199 of FIG. 1A during drilling or completion of the wellbore 120 for which example embodiments may be used. Referring to the description above with respect to FIGS. 1A through 1C, the detail of FIG. 2 shows a time, prior to casing operations discussed above, when fluids (e.g., drilling mud, completion fluid) are injected and/or lost downhole in the wellbore 120 and along the fractures 101 that emanate from the wellbore 120. These fluids interact with the subterranean formation, including grains 171 (e.g., small rock) adjacent to (e.g., in the secondary fractures) the frac face 102 of the fractures 101.

In addition to interacting with the grains 171 and other parts of the subterranean formation 110, these fluids interact with the fluids (e.g., formation water) already in the subterranean formation 110. As a result of all these interactions with and between the various fluids and parts of the subterranean formation 110, scale depositions 213 may form, such as in the porous media near the frac face 102, as shown in FIG. 2. To the extent that grains 171 and/or scale depositions 213 are located inside the fracture 101 formed by the frac face 102, one or more pore throats 119 may be defined. The time captured in FIG. 2 precedes the time captured in FIG. 1C.

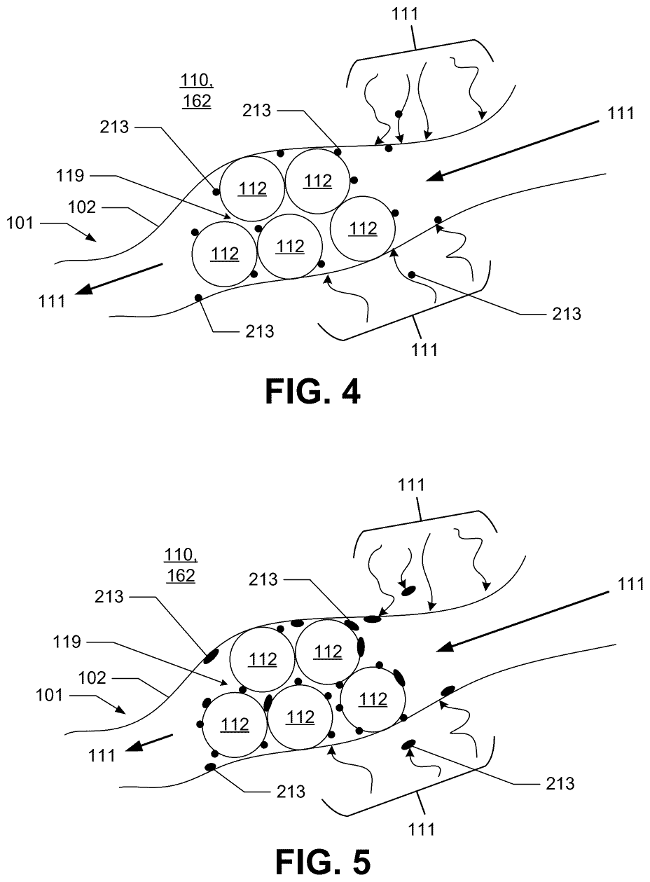

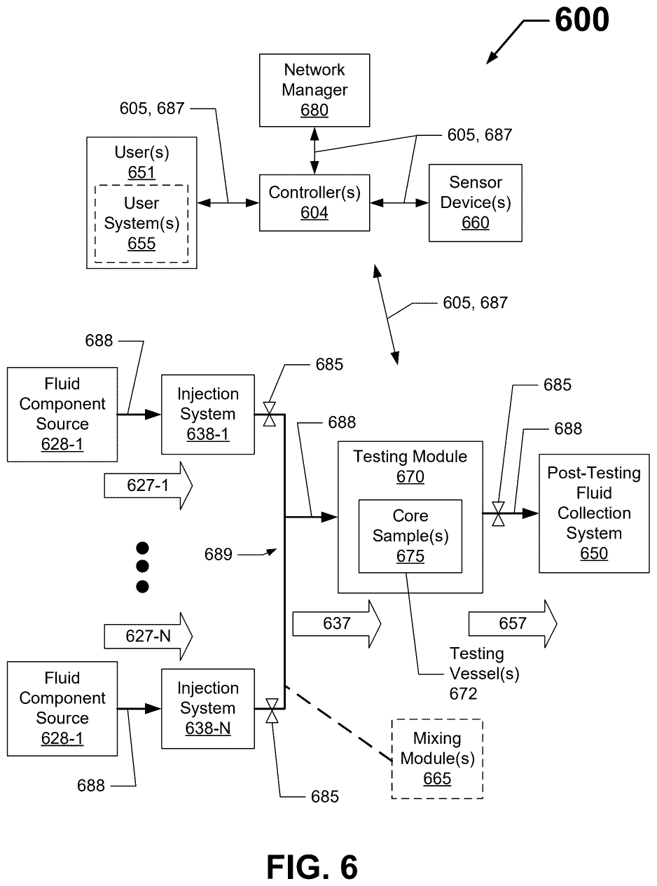

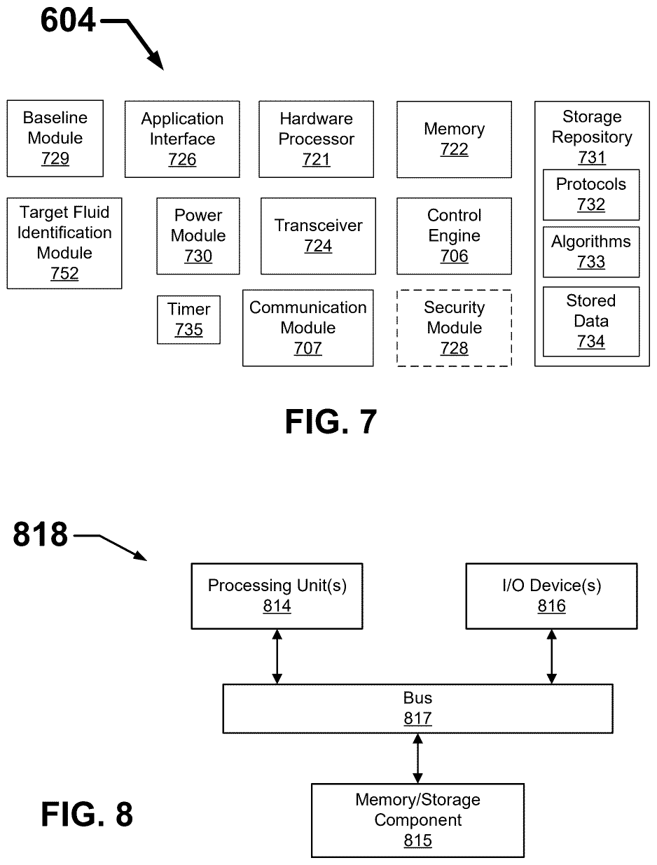

FIG. 3 shows the detail of a fracture 101 of the field system 199 of FIG. 1A during a shut-in period of the wellbore 120 for which example embodiments may be used. Referring to the description above with respect to FIGS. 1A through 2, the detail of FIG. 3 shows that when the wellbore 120 is shut in before fracturing operations, the rock-fluid interaction shown in FIG. 2 continues. As a result, formation of scale depositions 213 begin and/or continue to develop within the subterranean formation 110, such as within the primary factures 101 bounded by the frac faces 102. These scale depositions 213 may negatively impact (e.g., restrict, prevent) the release of subterranean resources 111 from the subterranean formation 110 into the wellbore 120. Also, the formation and/or accumulation of scale depositions 213 and/or other obstacles within the fracture 101 reduce the size of the pore throats 119. The time captured in FIG. 3 supersedes the time captured in FIG. 2 and precedes the time captured in FIG. 1C.FIG. 4 shows the detail of FIG. 1C at a subsequent point in time according to certain example embodiments. FIG. 5 shows the detail of FIG. 4 at a subsequent point in time according to certain example embodiments. For example, FIG. 4 may show the detail of FIG. 1C six months later than the time captured in FIG. 1C after flowing a scale enhancer (a type of fluid) therethrough, and FIG. 5 may show the detail of FIG. 4 four years later than the time captured in FIG. 4 after continuing to flow the scale enhancer therethrough. Referring to FIGS. 1A through 5, the detail in FIG. 4 shows, in addition to the proppant 112 within the fracture 101, a subterranean resource 111 (e.g., natural gas, oil) is shown flowing within the fracture 101 from the rock matrix 162, around the proppant 112 in the fracture 101, and on to the wellbore 120.

As the subterranean resource 111 flows within the paths formed by the rock matrices 162 and around or on the proppant 112 in the fracture 101, scale deposition 213 may occur (e.g., scale particles formed during the shut-in stage before the well is put on production) within the rock matrices 162, on the proppant 112, and/or on the frac face 102. (It should be noted that scale deposition 213 as defined herein may generally refer to any type of solid, which may also include, but is not limited to, asphaltenes, sludge, and fines.) Over time, the scale depositions 213 may begin to accumulate on the rock matrices 162, on the proppant 112, and/or on the frac face 102. The formation and/or accumulation of scale depositions 213 and/or other obstacles may change (e.g., reduce) the size of the pore throats 119. In some cases, at least some of the scale depositions 213 may be an inorganic deposit from ionic materials in water that attaches to solid surfaces. Hydrocarbons may be adsorbed on scale depositions 213. Under field conditions, scale depositions 213 may be a mixture of inorganic and organic components.

Scale depositions 213 may be initiated during a prior phase (e.g., completion) of a field operation, where fluids (outside of the fluids discussed below with respect to FIG. 6) and chemicals used downhole may interact with formation rock (e.g., the frac face 102, the rock matrices 162), resulting in the mobilization and release of elements from the rock matrices 162 adjacent to the fractures 101, and comingle with formation water in and/or near perforations and along fractures 101. Later, in a subsequent phase (e.g., shutting in) of the field operation, the rock-fluid interaction and the commingling of different fluids may lead to the formation (crystallization) and growth of scale depositions 213 in or near the perforations, the rock matrices 162, and the fractures 101. In yet another subsequent phase (e.g., production) of the field operation, the degradation in the conductivity and production flow path integrity over time in the rock matrices 162 and the fractures 101, caused by agglomerate build up of scale depositions 213, may lead to plugging (e.g., closing or highly restricting some or all of the pore throats 119) in or near the perforations, rock matrices 162, fractures 101, and completion tools.

The scale depositions 213 that accumulate within the rock matrices 162 and the fractures 101 may be composed of one or more of any of a number of items, including but not limited to calcium carbonate, barium sulfate, calcium sulfate, strontium sulfate, iron carbonate, iron oxide, iron sulfide, other oxides, other sulfides, other carbonates, other sulfates, halides, and hydroxides. While the scale depositions 213 may additionally or alternatively be composed of other items (e.g., gas hydrates, organic deposits (e.g., asphaltenes, waxes, acid induced sludges), and naphthenates), example embodiments may, in some cases, focus on the reduction of scale depositions 213 caused by inorganic deposits. The scale depositions 213 may be caused by one or more of any of a number of factors, including but not limited to supersaturation, mixing incompatible ions, changes in temperature, changes in pressure, carbon dioxide interaction, and a change in the pH of water in the fluid.

Scale depositions 213 may form during the shut-in stage prior to the well being put into production, as shown in FIG. 4. In such a case, the scale depositions 213 deposited on the rock matrices 162, on the proppant 112, and on the frac face 102 may be small and spotty. As a result, the scale depositions 213 do not contribute much to inhibiting the flow of the subterranean resource 111 through the paths within the rock matrices 162 and around the proppant 112 within the fracture 101 formed by the frac face 102. In the portion of the fracture 101 shown at the time captured in FIG. 4, there are 2 separate scale depositions 213 within the rock matrices 162, 8 scale depositions 213 on the proppant 112, and 4 scale depositions 213 on the frac face 102. The number, size, and location of the scale depositions 213 within the rock matrices 162 and the fracture 101 may vary.

When the well is put on production, some scale depositions 213 may stay at their original position, while some scale particles may move/migrate together with the produced water and deposit at another location along the production pathway. As more water is produced, if no mitigation efforts are made, the existing scale depositions 213 may increase in size and new scale depositions 213 may develop over time. As a result, the pore throats 119 may become highly restricted or closed. An example of this is captured in FIG. 5, which shows that the scale depositions 213 become larger and less spotty. As a result, the scale depositions 213 in FIG. 5 begin to contribute to reducing the size of the pore throats 119, inhibiting the flow of the subterranean resource 111 (e.g., a hydrocarbon) along the paths formed by the rock matrices 162, through the frac face 102 (impacting migration of the subterranean resource 111 from the rock matrix 162), and around the proppant 112 (combined with the scale depositions 213 on the proppant 112 and on the frac face 102) within the fracture 101.

In the portion of the fracture 101 shown at the time captured in FIG. 5, there are 25 separate scale depositions 213 within the rock matrices 162, at the frac face 102, and on the proppant 112, many of which are significantly larger than the size of the scale depositions 213 shown in FIG. 4. Also, some of the scale depositions 213 in FIG. 5 have migrated to a new location relative to their location in FIG. 4. Again, the number, size, and location of the scale depositions 213 within the fracture 101 may vary. Example embodiments may be designed in some cases to analyze the type of inorganic material in the scale depositions 213 in a particular experiment or field condition of a field operation. Example embodiments are also designed to determine the optimal way to reduce (e.g., remediate (e.g., removal of scale depositions 213 with a chemical treatment in the form of a fluid (e.g., an acid, a chelant)), mitigate) the development and accumulation of the scale depositions 213 in that particular field operation.

The formation of scale depositions 213 may have varying impacts on field operations, depending for instance on the formation type of the subterranean formation 110 in which the volume 190 is located. The following table provides an example of characteristics of three different formation types of the subterranean formation 110: a high-quality conventional reservoir, tight-gas sandstone, and shale. As discussed above, a pore throat 119 may include a passageway around obstacles (e.g., scale depositions 213, proppant 112) within a fracture 101. More obstacles that are positioned within a fracture 101 result in more pore throats 119 in the fracture 101. As the table shows, scale has a larger impact on unconventional formations rather than conventional formations. Example embodiments are designed to identify and develop methods to control the formation of scale depositions 213 at the subsurface (e.g., in porous media near the frac face 102, in the pore throats 119) in a manner that eliminates and/or minimizes the negative impact of subsurface scale depositions 213 on recovery of hydrocarbon resources 111 and/or estimated ultimate recovery (EUR) from the wellbore 120.

| | High-Quality | | |
|---|---|---|---|
| | Conventional | Tight-Gas | |
| Type of Reservoir/Formation | Reservoir | Sandstone | Shale |
| Pore Throat Size | >10 | 0.03 to 2.00 | 0.005 to 0.1 |
| (Diameter in μm) | | | |
| Pore Throat Size to Barite | >11256 | 34 to 2251 | 6 to 113 |
| Unit Cell Length Ratio | | | |
| Impact of Scale Deposition | Low | High | Very High |
| on Hydrocarbon Recovery | | | |

FIG. 6 shows a diagram of a testing system 600 for assessing and reducing deposition of scales and/or other solids according to certain example embodiments. The testing system 600 of FIG. 6 includes one or more fluid component sources 628, one or more injection systems 638, a testing module 670, a post-testing fluid collection system 650, one or more optional mixing modules 665, one or more controllers 604, one or more sensor devices 660, one or more users 651 (including one or more optional user systems 655), a network manager 680, piping 688, and one or more valves 685. The testing module 670 includes one or more testing vessels 672.

The components shown in FIG. 6 are not exhaustive, and in some embodiments, one or more of the components shown in FIG. 6 may not be included in the example testing system 600. Any component of the testing system 600 may be discrete or combined with one or more other components of the testing system 600. Also, one or more components of the testing system 600 may have different configurations. For example, one or more sensor devices 660 may be disposed within or disposed on other components (e.g., the piping 688, a valve 685, the testing module 670, the post-testing fluid collection system 650). As another example, a controller 604, rather than being a stand-alone device, may be part of one or more other components (e.g., testing module 670, the post-testing fluid collection system 650, an injection system 638) of the testing system 600.

Referring to the description above with respect to FIGS. 1A through 5, the testing system 600 of FIG. 6 may be designed to simulate waterflooding, which is a secondary recovery method used in deepwater oil fields. Waterflooding commonly uses seawater as a cost-effective source of injection water. However, the high sulfate content (e.g., approximately 2,775 mg/L) of untreated seawater (also sometimes called high sulfate seawater (HSSW)) can react with formation water ions (e.g., calcium, barium, strontium), increasing the risk of sulfate scale precipitation and associated operational challenges. For example, introducing sulfate-rich seawater into formation brines containing elevated barium and strontium concentrations can promote the precipitation of barite (BaSO₄) and celestite (SrSO₄). These sulfate scales have the potential to reduce permeability, impair well productivity, and precipitate in the near-wellbore region, production tubing, and topside facilities following seawater breakthrough, as discussed below with respect to FIGS. 2 through 5. Scaling is primarily triggered by mixing incompatible formation water with injected seawater, making proactive scale management essential for long-term production efficiency.

The testing module 670 of the testing system 600 is designed to evaluate the potential for natural mitigation of sulfate scale (e.g., produced by seawater injection) through water-rock interactions, as observed in controlled coreflood experiments. For example, the testing module 670 of FIG. 6 may quantify changes in SO₄ levels resulting from rock-fluid-fluid interactions, thereby supporting an injection scheme for secondary recovery that reduces or eliminates the need for field equipment (e.g., field equipment 109) such as Sulfate Removal Units (SRUs) from field development requirements. While effective, SRUs substantially increase capital expenditures (CAPEX) and operational expenditures (OPEX), and their installation often becomes a major economic hurdle, especially during industry downturns when budget constraints intensify. As a result, operators seek alternatives or supporting mechanisms that can reduce or postpone the need for SRUs.

One of the purposes of the testing module 670 of the testing system 600 of FIG. 6 is to explore viable options for in-situ sulfate stripping, where water-rock interactions deep within the reservoir can significantly reduce the sulfate concentrations in the water prior to its breakthrough at the production wells. This process can significantly decrease the sulfate concentration arriving at producers and consequently reduce the scaling tendency. The example testing module 670 uses core samples 675 (also sometimes known as core plugs) from one or more particular wellbores in a field system to generate a detailed understanding of the in-situ sulfate stripping mechanism that may be used, allowing for a balancing of technical scale management requirements (and associated costs) with the financial implications of SRU installation used in the current art.

With the testing system 600 of FIG. 6, a fluid 637 is pushed through one or more testing vessels 672 of the testing module 670. As defined herein, a fluid 637 is a liquid in aqueous phase. Examples of a fluid 637 may be or include, but are not limited to, produced water (with or without chemical additives), injection water, produced fluids (e.g., oil, water), aqueous fluids prepared in a lab or received from an oilfield/well, synthesized brine, and chemical products (e.g., diluted liquid chemical products, non-diluted liquid chemical products). A fluid 637 is made up of multiple fluid components 627 (e.g., water, a dissolved salt, a chelant, a cation, an anion, a scale inhibitor additive, a brine) that are mixed together before reaching the testing module 670.

Two or more fluid components 627 may be mixed together in the piping 688 at a header 689 as those fluid components 627 interact with each other to form a fluid 637 and flow toward the testing module 670. Alternatively, the testing system 600 may include one or more of the optional mixing modules 665 that mix two or more fluid components 627 together before the fluid components 627 reach the testing module 670 as a fluid 637. A mixing module 665 may be or include one or more of a number of features used to mix two or more fluid components 627 together. Such features may include, but are not limited to, a vessel, a sensor device 660, a controller 604, an agitator, a paddle, a circulating system, an aerator, a vibrating mechanism, and a centrifuge. A mixing module 665 and the header 689 may be part of a common vessel herein.

There may be one or more fluid component sources 628. In certain example embodiments, there are at least two fluid component sources 628. As shown in FIG. 6, the testing system 600 includes fluid component source 628-1 (which holds fluid component 627-1) through fluid component source 628-N (which holds fluid component 627-N). Each fluid component 627 (e.g., an additive) may be or include a fluid. A single fluid component 627 or a mixture of multiple fluid components 627 (but not the fluid 637) may be disposed in a fluid component source 628. In certain example embodiments, when a fluid 637 is or includes an anionic brine, two fluid component sources 628 may be or include NaCl and NaHCO₃, each of which may be dissolved in de-ionized (DI) water. When the scale depositions 213 include calcite, the anion HCO₃, which originates from the NaHCO₃ salt, is included in the brine to provide formation of the calcite scale depositions 213. Even though this fluid 637 includes both cations and anions, it is called an anionic brine because of the HCO₃.

In addition, or in the alternative, the fluid may be or include a cationic brine (Ca²⁺). In such a case, two fluid component sources 628 may be or include NaCl and CaCl₂, each of which may be dissolved in DI water. When the scale depositions 213 includes calcite, the cation Ca²⁺ which originates from the CaCl₂ salt, is included in the brine to provide formation of the calcite scale depositions 213. Even though this fluid 637 includes both cations and anions, it is called a cationic brine because of the Ca²⁺.

To control the composition of the fluid 637 at a given point in time, the amount of the individual fluid components 627 that are released or withdrawn from a fluid component source 628 may be regulated in real time. This regulation may be performed automatically by a controller 604 or manually by a user 651 (including an associated user system 655). This regulation may be performed using equipment such as the injection systems 638, valves 685, regulators, sensor devices 660, and meters. Examples of a fluid component source 628 may include, but are not limited to, a natural vessel (e.g., land that forms a natural body of water) and a man-made storage tank or other vessel. A fluid component 627 of a fluid component source 628 may have any of a number of different compositions that are naturally occurring or man-made. In some cases, a fluid component 627 of the fluid 637 includes water.

Each injection system 638 is configured to extract a fluid component 627 from a fluid component source 628 and push the fluid component 627 toward the testing module 670. The number of injection systems 638 in the testing system 600 may vary. In this case, there are N injection systems 638 (injection system 638-1 through injection system 638-N). In some embodiments, there may be one injection system 638 for each fluid component source 628. In alternative embodiments, there may be one injection system 638 for multiple fluid component sources 628. Each injection system 638 may include one or more of a number of pieces of equipment to perform its function. Examples of such equipment may include, but are not limited to, a compressor, a motor, a pump, a heater, a fan, a blower, piping (e.g., piping 688), a valve (e.g., valve 685), a controller (e.g., controller 604), and a sensor device (e.g., sensor device 660).

The piping 688 (including the header 689) may include multiple pipes, ducts, elbows, joints, sleeves, collars, and similar components that are coupled to each other (e.g., using coupling features such as mating threads) to establish a network for transporting the fluid components 627 from the fluid component sources 628, through the injection systems 638, to the header 689 (where the fluid components 627 mix together to form a fluid 637), to the testing module 670, and finally from the testing module 670 to the post-testing fluid collection system 650. Each component of the piping 688 may have an appropriate size (e.g., inner diameter, outer diameter) and be made of an appropriate material (e.g., steel, PVC) to safely and efficiently handle the pressure, temperature, flow rate, and other characteristics of the fluid components 627 or each fluid 637, as applicable.

There may be a number of valves 685 placed in-line with the piping 688 at various locations (including at the header 689) in the testing system 600 to control the flow of fluid components 627 and/or each fluid 637 therethrough. A valve 685 may have one or more of any of a number of configurations, including but not limited to a guillotine valve, a ball valve, a gate valve, a butterfly valve, a pinch valve, a needle valve, a plug valve, a diaphragm valve, and a globe valve. One valve 685 may be configured the same as or differently compared to another valve 685 in the testing system 600. Also, one valve 685 may be controlled (e.g., manually, automatically by the controller 604) the same as or differently compared to another valve 685 in the testing system 600.

The testing module 670 is configured to house one or more testing vessels 672. The testing module 670 receives a fluid 637 from the header 689, allows the fluid 637 to run through one or more testing vessels 672, and sends the post-testing fluid 657 to the post-testing fluid collection system 650. When multiple testing vessels 672 are involved in a particular test with a fluid 637, one testing vessel 672 may be configured in series and/or in parallel with respect to one or more of the other testing vessels 672.

In certain example embodiments, the testing vessels 672 are passive objects that have a fluid 637 pass through them without the testing vessels 672 being modified or taking action during this process. In such a case, the testing module 670 may control various aspects (e.g., temperature, pressure, flow rate) of the fluid 637 and/or the testing vessel 672. In certain example embodiments, the testing module 670 is designed to subject a core sample 675 in the testing vessel 672 to conditions (e.g., pressure, temperature, flow rate) that are representative of the corresponding conditions of the fractures 101 and rock matrices 162 in the subterranean formation 110 adjacent to the wellbore 120. The testing module 670 may include one or more of a number of pieces of equipment to perform these functions. Examples of such equipment may include, but are not limited to, a motor, a pump, a compressor, a heater, a fan, a blower, piping (e.g., piping 688), a valve (e.g., valve 685), a controller (e.g., controller 604), and a sensor device (e.g., sensor device 660).

A testing vessel 672 is a vessel (e.g., a column) inside of which one or more core samples 675 are disposed. A core sample 675 in a testing vessel 672 is extracted from the wellbore 120 and may include fractures 101 in a subterranean formation 110 adjacent to the wellbore 120. In some cases, a core sample 675 is extracted from the subterranean formation 110 by a tool (e.g., a coring tool, a wireline tool) placed in the wellbore 120 adjacent to the fractures 101. In such a case, the core sample 675 may be retrieved from the tool when the tool is brought to the surface 108 and subsequently placed, either intact or crushed (cutting size), in a testing vessel 672. In some cases, a core sample 675 may include proppant 112 used to prop open the fractures 101.

A core sample 675 may be defined by one or more factors. Examples of factors that may be controlled with respect to a core sample 675 in a testing vessel 672 may include, but are not limited to, the number of core samples 675, the content (e.g., rock, materials (e.g., metal) of field equipment, proppant 112, scale inhibitor, oil-phase solids, oil-phase sludges, water-phase solids, water-phase sludges) of a core sample 675, the size of a core sample 675, and the shape (e.g., cylindrical) of a core sample 675. In some cases, a core sample 675 is prepared in some way. For example, a core sample 675 may be fully or partially covered in a nickel alloy mesh that is secured to the core sample 675 using polytetrafluoroethylene (PTFE) tape.

The main purpose of the testing module 670 is to control the conditions under which a core sample 675 is exposed before, during, and/or after testing of the core sample 675. For example, the testing module 670 may replicate downhole conditions by continually providing a fluid 637 that flows through the core sample 675 in the testing vessel 672. In order to accomplish this, the testing vessel 672 may be made of any of a number of appropriate materials (e.g., glass, polytetrafluoroethylene-lined stainless steel) that may withstand the conditions (e.g., pressure, temperature, salinity, flow rate) experienced by the testing module 670, which are designed to be representative of downhole conditions.

Testing a core sample 675 in the testing vessel 672 of the testing module 670 may be or include a coreflood test. After a period of time, the testing process may be paused or stopped so that the core sample 675 in the testing vessel 672 may be evaluated. In some example embodiments, the fluid 637 may be designed to reduce (e.g., eliminate, lower) scaling that may appear and grow on some of the core sample 675 (e.g., the proppant 112, rock, materials (e.g., metals) representative of downhole equipment (e.g., casing pipe) and/or other (e.g., surface) equipment (e.g., wellhead, pumping equipment) used in a field operation) in the testing vessel 672.

Evaluation of the core sample 675 in the testing vessel 672 may include characterizing (e.g., determining the amount of) scale depositions 213 disposed on the proppant 112, rock, and/or other core sample 675 in a testing vessel 672 over time. This characterization and evaluation may then be correlated to how a fluid 637 that includes a scale inhibitor or other fluids/chemicals used during that phase of testing may effect flow rate through the pore throats 119 within the fractures 101 in the core sample 675 and how the flow rate correlates to controlling (e.g., eliminating, reducing, maintaining, increasing) scale depositions 213 in the fractures 101 in the core sample 675. Results that are achieved with the core samples 675 may be replicated in the wellbore 120.

As another example, if a desired goal is to use the testing module 670 to determine the impact of freshly formed scale depositions 213 on fracture conductivity, the testing module 670 may be used to gauge the optimal fluid 637 (e.g., the concentration of a particular brine) so that the formation of scale depositions 213 occurs on some or all of the core sample 675 in the testing vessel 672. For instance, an initial test may be performed to determine the amount of time (sometimes called induction time) it takes for calcite (a form of scale deposition 213) to start to form. By mixing a fluid of cationic (Ca²⁺) and anionic (HCO₃²⁻) brines (individually, these brines are considered fluid components 627 of the fluid 637), it may be found that scale depositions 213 develop after 40 seconds in a test tube or bottle (a form of testing vessel 672). As yet another example, if a desired goal is to use the testing module 670 to determine if a non-scaling fluid 637 (e.g., a type of brine) may pass through the core sample 675 in the testing vessel 672 without disturbing the proppant 112, the testing module 670 may be used to demonstrate blockage within the core sample 675 in the testing vessel 672 using a fluid 637 that promotes scaling.

As still another example, if a desired goal is to use the testing module 670 to demonstrate that a particular fluid 637 (also sometimes called a chemical treatment herein) may decrease formation of scale depositions 213 and other blockage in the core sample 675 (or components thereof, such as proppant 112 and rock) within the testing vessel 672, the testing module 670 may be used to analyze the effectiveness of various fluids 637 as scale inhibitors. In some cases, the testing module 670 may include one or more features (e.g., a spectrograph, a gas chromatograph, a camera with a high zoom lens, a controller 604, one or more sensor devices 660) that perform some or all of the evaluation of core sample 675 within a testing vessel 672 that have been tested. The testing vessel 672 may be removable (e.g., by a user 651) from and insertable into the testing module 670. The testing module 670 may include one or more features (e.g., a clamp, a latched lid) that ensure that a testing vessel 672 is secure within the testing module 670.

Objectives that may be achieved by having a fluid 637 flow through a core sample 675 in a testing vessel 672 of the testing module 670 may include, but are not limited to, determining whether scale depositions 213 may deposit at subsurface fractures, determining how scale deposition 213 on the core sample 675 impacts permeability and fluid flow, determining how scale deposition 213 on the core sample 675 impacts the frac face 102 and surface of proppant 112, determining how much scale deposition 213 may cause significant change in permeability, determining how adding scale inhibitor in a fluid 637 (e.g., a scaling brine) may mitigate scale depositions 213 on the core sample 675, determining the impact of crushing/embedding/clustering of proppant 112 on solid depositions (e.g., scale depositions 213) and flow assurance risks (e.g., plugging, fluid flow restriction), determining the effectiveness and impact of chemical additives (e.g., chelants, acids) as fluid components 627 of a fluid 637 on the removal of scale depositions 213 at fractures 101 (e.g., in rock matrices 162, on proppant 112, on a frac face 102), determining the effectiveness of pre-packed solid scale inhibitors as fluid components 627 of a fluid 637 in mitigating scale deposition 213 from produced water, studying adsorption and desorption of scale depositions 213 from a frac face 102 in fractures 101, optimizing squeeze treatment design to control scale depositions 213, and determining the impact of water cut (representative of field condition produced fluid contains both oil and water) on scale depositions 213 on a frac face 102, rock matrices 162, and proppants 112.

Each sensor device 660 includes one or more sensors that measure one or more parameters (e.g., pressure, flow rate, temperature, humidity, fluid content, voltage, current, permeability, porosity, rock characteristics, pH value, morphology, crystal structure, scale type, material composition). Examples of a sensor of a sensor device 660 may include, but are not limited to, a temperature sensor, a flow sensor, a pressure sensor, a gas spectrometer, a chromatograph (e.g., an ion chromatograph (IC), a gas chromatograph (GC)), an X-ray fluorescence (XRF) device, a quantitative X-ray diffraction (QXRD) device, an Inductively Coupled Plasma (ICP) device, a mass spectrometer (MS), an optical emission spectrometer (OES), a pH meter, a particle size analyzer, a scanning electron microscope (SEM), an infrared transmitter and/or receiver, a voltmeter, an ammeter, a permeability meter, a porosimeter, and a camera. A sensor device 660 may be integrated with or measure a parameter associated with one or more components of the testing system 600. For example, a sensor device 660 may be configured to measure a parameter (e.g., flow rate, pressure, temperature) of a fluid component 627, a fluid 637, and/or a post-testing fluid 657 flowing through the piping 688 at a particular location (e.g., between a fluid component source 628 and a corresponding injection system 638, between the header 689 and the testing module 670, between the testing module 670 and the post-testing fluid collection system 650).

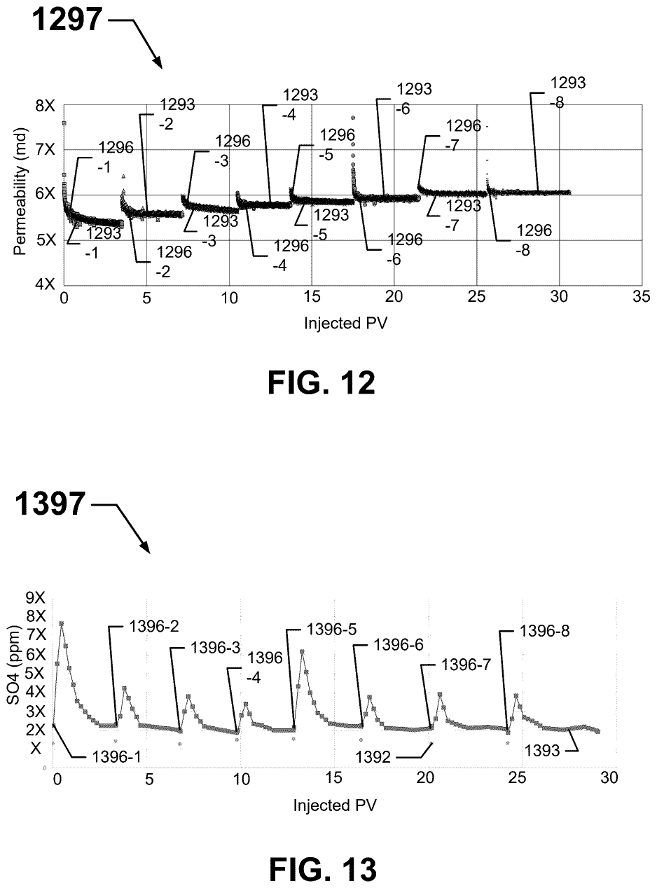

As another example, a sensor device 660 may be configured to determine how open or closed a valve 685 within the testing system 600 is. As yet another example, one or more sensor devices 660 may be used to characterize (e.g., identify an amount of) scale depositions 213 that have accumulated on proppant 112 and/or a frac face 102 of a fracture 101 within a core sample 675 in a testing vessel 672. In some cases, a number of sensor devices 660, each measuring a different parameter, may be used in combination to determine and confirm whether a controller 604 should take a particular action (e.g., operate a valve 685, operate or adjust the operation of the testing module 670). When a sensor device 660 includes its own controller 604 (or portions thereof), then the sensor device 660 may be considered a type of computer device, as discussed below with respect to FIG. 8.

One or more sensor devices 660 may be integrated with the testing module 670. For example, two sensor devices 660 in the form of or including pressure sensors may be positioned before the testing vessel 672 and after the testing vessel 672 to provide a differential pressure value across the testing vessel 672. The differential pressure value may provide information as to, for example, a change in permeability, an accumulation of scale depositions 213, and/or other plugging in the core sample 675. In addition, or in the alternative, one or more sensor devices 660 (e.g., a permeability meter) may be integrated with the testing vessel 672 to measure the permeability of the core sample 675. In some cases, in order to ensure that the post-testing fluid collection system 650 receives the post-testing fluid 657 from the testing module 670 at an appropriate pressure, a sensor device 660 in the form of a pressure regulator (or other similar equipment) may be installed between the testing vessel 672 and the post-testing fluid collection system 650.

In certain example embodiments, the post-testing fluid collection system 650 is configured to receive the post-testing fluid 657, which is the byproduct of the fluid 637 that has flowed through and/or otherwise interacted with the core sample 675 in one or more testing vessels 672 of the testing module 670. The post-testing fluid collection system 650 may include a vessel (e.g., a tank, a flask, a column) to contain some or all of the post-testing fluid 657. In some cases, the post-testing fluid collection system 650 may also be configured to perform one or more tests on the post-testing fluid 657. In such cases, the post-testing fluid collection system 650 may include one or more of a number of features (e.g., a motor, a pump, a compressor, a heater, a fan, a blower, piping (e.g., piping 688), a valve (e.g., valve 685), one or more sensor devices 660 (e.g., a spectrograph, a gas chromatograph, a camera with a high zoom lens), a controller 604) to conduct such testing.

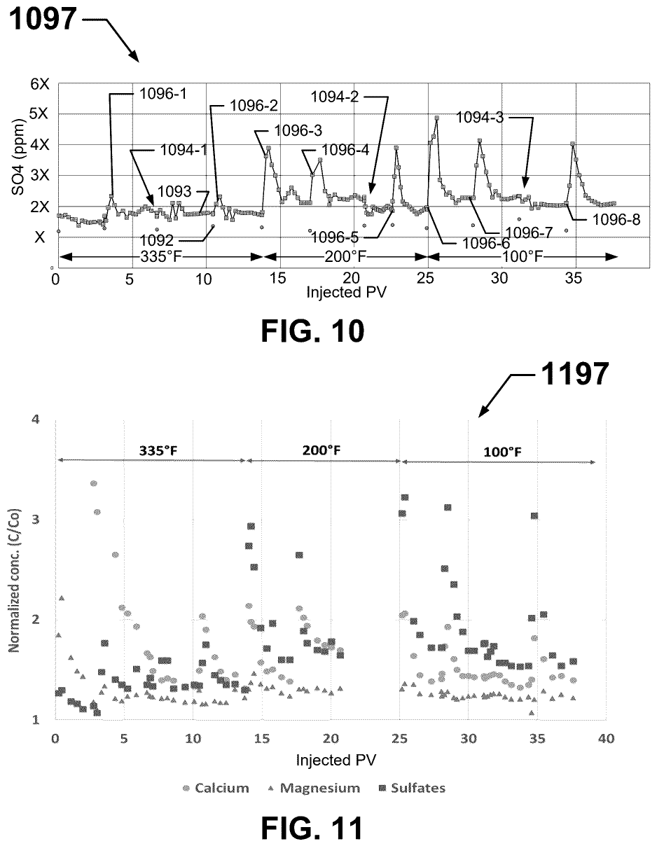

The testing system 600 may include one or more controllers 604. A controller 604 of the testing system 600 communicates with and in some cases controls one or more of the other components (e.g., a sensor device 660, an injection system 638, the testing module 670, the post-testing fluid collection system 650) of the testing system 600. A controller 604 performs a number of functions that include obtaining and sending data, evaluating data, following protocols, running algorithms, and sending commands. A controller 604 may include one or more of a number of components. As discussed below with respect to FIG. 7, such components of a controller 604 may include, but are not limited to, a control engine, a communication module, a timer, a power module, a storage repository, a hardware processor, memory, a transceiver, an application interface, and a security module.

When there are multiple controllers 604 (e.g., one controller 604 for one or more injection systems 638, another controller 604 for the testing module 670, yet another controller 604 for the post-testing fluid collection system 650), each controller 604 may operate independently of each other. Alternatively, one or more of the controllers 604 may work cooperatively with each other. As yet another alternative, one of the controllers 604 may control some or all of one or more other controllers 604 in the testing system 600. Each controller 604 may be considered a type of computer device, as discussed below with respect to FIG. 8.

A user 651 may be any person that interacts, directly or indirectly, with a controller 604 and/or any other component of the testing system 600. Examples of a user 651 may include, but are not limited to, a business owner, a research scientist, an engineer, a company representative, a geologist, a consultant, a drilling engineer, a contractor, and a manufacturer's representative. A user 651 may use one or more user systems 655, which may include a display (e.g., a GUI). A user system 655 of a user 651 may interact with (e.g., send data to, obtain data from) the controller 604 via an application interface and using the communication links 605. The user 651 may also interact directly with the controller 604 through a user interface (e.g., keyboard, mouse, touchscreen).

The network manager 680 is a device or component that controls all or a portion (e.g., a communication network, the controller 604) of the testing system 600. The network manager 680 may be substantially similar to the controller 604, as described above. For example, the network manager 680 may include a controller that has one or more components and/or similar functionality to some or all of the controller 604. Alternatively, the network manager 680 may include one or more of a number of features in addition to, or altered from, the features of the controller 604. As described herein, control and/or communication with the network manager 680 may include communicating with one or more other components of the same testing system 600 or another system. In such a case, the network manager 680 may facilitate such control and/or communication. The network manager 680 may be called by other names, including but not limited to a master controller, a network controller, and an enterprise manager. The network manager 680 may be considered a type of computer device, as discussed below with respect to FIG. 8.

Interaction between each controller 604, the sensor devices 660, the users 651 (including any associated user systems 655), the network manager 680, and other components (e.g., the valves 685, an injection system 638, the testing module 670, the post-testing fluid collection system 650) of the testing system 600 may be conducted using communication links 605 and/or power transfer links 687. Each communication link 605 may include wired (e.g., Class 1 electrical cables, Class 2 electrical cables, electrical connectors, Power Line Carrier, RS685) and/or wireless (e.g., Wi-Fi, Zigbee, visible light communication, cellular networking, Bluetooth, Bluetooth Low Energy (BLE), ultrawide band (UWB), WirelessHART, ISA100) technology. A communication link 605 may transmit signals (e.g., communication signals, control signals, data) between each controller 604, the sensor devices 660, the users 651 (including any associated user systems 655), the network manager 680, and the other components of the testing system 600.

Each power transfer link 687 may include one or more electrical conductors, which may be individual or part of one or more electrical cables. In some cases, as with inductive power, power may be transferred wirelessly using power transfer links 687. A power transfer link 687 may transmit power between each controller 604, the sensor devices 660, the users 651 (including any associated user systems 655), the network manager 680, and the other components of the testing system 600. Each power transfer link 687 may be sized (e.g., 12 gauge, 18 gauge, 4 gauge) in a manner suitable for the amount (e.g., 680V, 24V, 120V) and type (e.g., alternating current, direct current) of power transferred therethrough.

FIG. 7 shows a system diagram of a controller 604 of FIG. 6 according to certain example embodiments. Referring to FIGS. 1A through 5, the controller 604 includes multiple components. In this case, the controller 604 of FIG. 7 includes a control engine 706, a communication module 707, a timer 735, a power module 730, a storage repository 731, a hardware processor 721, a memory 722, a transceiver 724, an application interface 726, a baseline module 729, a target fluid identification module 752, and, optionally, a security module 728. The controller 604 (or components thereof) may be located at or near the various components of the testing system 600. In addition, or in the alternative, the controller 604 (or components thereof) may be located remotely from (e.g., in the cloud, at an office building) the various components of the testing system 600.

The storage repository 731 may be a persistent storage device (or set of devices) that stores software and data used to assist the controller 604 in communicating with one or more other components of a system, such as the users 651 (including associated user systems 655), each injection system 638, the testing module 670, each post-testing fluid collection system 650, the network manager 680, the sensor devices 660, and any other component of the testing system 600 of FIG. 6 above. In one or more example embodiments, the storage repository 731 stores one or more protocols 732, algorithms 733, and stored data 734.

The protocols 732 of the storage repository 731 may be any procedures (e.g., a series of method steps) and/or other similar operational processes that the control engine 706 of the controller 604 follows based on certain conditions at a point in time. The protocols 732 may include any of a number of communication protocols that are used to send and/or obtain data between the controller 604 and other components of a system (e.g., testing system 600). Such protocols 732 used for communication may be a time-synchronized protocol. Examples of such time-synchronized protocols may include, but are not limited to, a highway addressable remote transducer (HART) protocol, a wirelessHART protocol, and an International Society of Automation (ISA) 100 protocol. In this way, one or more of the protocols 732 may provide a layer of security to the data transferred within a system (e.g., testing system 600). Other protocols 732 used for communication may be associated with the use of Wi-Fi, Zigbee, visible light communication (VLC), cellular networking, BLE, UWB, and Bluetooth.

The algorithms 733 may be any formulas, mathematical models, forecasts, simulations, and/or other similar tools that the control engine 706 of the controller 604 uses to reach a computational conclusion. For example, one or more algorithms 733 may be used, in conjunction with one or more protocols 732, to assist the controller 604 to determine when to start, adjust, and/or stop the operation of the testing module 670 and/or the post-testing fluid collection system 650. As another example, one or more algorithms 733 may be used, in conjunction with one or more protocols 732, to assist the controller 604 to determine when to start, adjust, and/or stop the operation of an injection system 638. As yet another example, one or more algorithms 733 may be used, in conjunction with one or more protocols 732, to assist the controller 604 to identify an optimal formulation of a fluid to reduce or eliminate scale depositions 213 on proppant 112 within a testing vessel 672. As still another example, one or more algorithms 733 may be used, in conjunction with one or more protocols 732, to assist the controller 604 in trending the performance of a fluid under certain conditions over time.

An example of an algorithm 733 is represented by the formula: Q=[kA(Pᵢ−Pₒ)]÷μL, where Q is a flow rate (in cm³/s), Pᵢ is inlet fluid pressure (in Pa), Pₒ is outlet fluid pressure (in Pa), μ is dynamic viscosity of the fluid (poise or Pa·S), L is the length of the core sample 675 in the testing vessel 672 (in cm), k is the permeability of the core sample 675 in the testing vessel 672 (in mD), and A is the area of the core sample 675 in the testing vessel 672 (in cm²).

Stored data 734 may be any data associated with a field (e.g., the subterranean formation 110, the fractures 101, the rock matrices 162 within the volume 190 adjacent to a wellbore 120, the characteristics of proppant 112 used in a field operation), other fields (e.g., other wellbores and subterranean formations), the other components (e.g., the user systems 655, the testing module 670, the core sample 675 in the testing vessel 672, the post-testing fluid collection system 650), including associated equipment (e.g., motors, pumps, compressors), of the testing system 600, measurements made by the sensor devices 660, threshold values, tables, results of previously run or calculated algorithms 733, updates to protocols 732, user preferences, and/or any other suitable data. Such data may be any type of data, including but not limited to historical data, present data, and future data (e.g., forecasts). The stored data 734 may be associated with some measurement of time derived, for example, from the timer 735.

Examples of a storage repository 731 may include, but are not limited to, a database (or a number of databases), a file system, cloud-based storage, a hard drive, flash memory, some other form of solid-state data storage, or any suitable combination thereof. The storage repository 731 may be located on multiple physical machines, each storing all or a portion of the communication protocols 732, the algorithms 733, and/or the stored data 734 according to some example embodiments. Each storage unit or device may be physically located in the same or in a different geographic location.

The storage repository 731 may be operatively connected to the control engine 706. In one or more example embodiments, the control engine 706 includes functionality to communicate with the users 651 (including associated user systems 655), the sensor devices 660, the network manager 680, and the other components in the testing system 600. More specifically, the control engine 706 sends information to and/or obtains information from the storage repository 731 in order to communicate with the users 651 (including associated user systems 655), the sensor devices 660, the network manager 680, and the other components of the testing system 600. As discussed below, the storage repository 731 may also be operatively connected to the communication module 707 in certain example embodiments.

In certain example embodiments, the control engine 706 of the controller 604 controls the operation of one or more components (e.g., the communication module 707, the timer 735, the transceiver 724) of the controller 604. For example, the control engine 706 may activate the communication module 707 when the communication module 707 is in “sleep” mode and when the communication module 707 is needed to send data obtained from another component (e.g., a sensor device 660) in the testing system 600. In addition, the control engine 706 of the controller 604 may control the operation of one or more other components (e.g., the testing module 670, the post-testing fluid collection system 650, an injection system 638), or portions thereof, of the testing system 600.

The control engine 706 of the controller 604 may communicate with one or more other components of the testing system 600. For example, the control engine 706 may use one or more protocols 732 to facilitate communication with the sensor devices 660 to obtain data (e.g., measurements of various parameters, such as temperature, pressure, and flow rate), whether in real time or on a periodic basis and/or to instruct a sensor device 660 to take a measurement. The control engine 706 may use measurements of parameters taken by sensor devices 660 while a fluid flows through the core sample 675 in a testing vessel 672, as well as one or more protocols 732 and/or algorithms 733, to analyze the performance of the fluid 637 (e.g., that includes a scale inhibitor with a concentration ranging from lower (e.g., 1 ppmv) concentrations to higher (e.g., 50 ppmv, up to 20%) concentrations) in reducing scale depositions 213 on proppant 112, rock, and/or other components of the core sample 675 in the testing vessel 672.

As yet another example, the control engine 706 may use one or more algorithms 733 and/or protocols 732 to recommend a change to the formulation (e.g., adding a fluid component 627, removing a fluid component 627, increasing an amount of a fluid component 627, decreasing an amount of a fluid component 627) of a fluid 637 in an attempt to improve reduction of scale depositions 213 on some or all of the core sample 675. For instance, a fluid 637 may include a scale inhibitor to prevent/inhibit the formation of new scale depositions 213 from an aqueous phase. As another example, a fluid 637 may include chelants, an acid treatment product, or a scale removal product to remove existing scale depositions 213. The testing system 600 may be used for either or both purposes. As a specific example, the core sample 675 may include proppant 112. An initial fluid 637 that flows through the core sample 675 in the testing vessel 672 of the testing module 670 may cause scale depositions 213 to form on the proppant 112. Later, a different fluid 637 that includes a non-scaling brine (e.g., a cation brine only) may flow through the core sample 675 in the testing vessel 672 of the testing module 670 to understand how the permeability of some or all of the core sample 675 evolves over time.

The control engine 706 may generate and process data associated with control, communication, and/or other signals sent to and obtained from the users 651 (including associated user systems 655), the sensor devices 660, the network manager 680, and the other components of the testing system 600. In certain embodiments, the control engine 706 of the controller 604 may communicate with one or more components of a system external to the testing system 600. For example, the control engine 706 may interact with an inventory management system by ordering replacements for components or pieces of equipment (e.g., a sensor device 660, a valve 685, a motor) within the testing system 600 that has failed or is failing. As another example, the control engine 706 may interact with a contractor or workforce scheduling system by arranging for the labor needed to replace a component or piece of equipment in the testing system 600. In this way and in other ways, the controller 604 is capable of performing a number of functions beyond what could reasonably be considered a routine task.

In certain example embodiments, the control engine 706 may include an interface (part of the application interface 726) that enables the control engine 706 to communicate with the sensor devices 660, the user systems 655, the network manager 680, and the other components of the testing system 600. For example, if a user system 655 operates under IEC Standard 62386, then the user system 655 may have a serial communication interface that will transfer data to the controller 604. Such an interface may operate in conjunction with, or independently of, the protocols 732 used to communicate between the controller 604 and the users 651 (including corresponding user systems 655), the sensor devices 660, the network manager 680, and the other components of the testing system 600.

The control engine 706 and/or other components of the controller 604 may also include one or more hardware components and/or software elements to perform its functions. Such components may include, but are not limited to, a universal asynchronous receiver/transmitter (UART), a serial peripheral interface (SPI), a direct-attached capacity (DAC) storage device, an analog-to-digital converter, an inter-integrated circuit (I2C), and a pulse width modulator (PWM).

The baseline module 729 of the controller 604 is configured to establish and/or maintain one or more baselines of one or more parameters. A baseline is used as a means of comparison for future data (e.g., measurements from sensor devices 660, outputs from algorithms 733) in order to reach a conclusion (e.g., find a target fluid). Each baseline established and/or maintained by the baseline module 729 is based on measurements made by one or more sensor devices 660 while testing a core sample 675 in the testing module 670 under certain conditions (e.g., using a fluid 637 with no scaling potential, using a fluid 637 with some amount of salt). The baseline module 729 may operate using one or more protocols 732, one or more algorithms 733, and/or stored data 734.

The target fluid identification module 752 of the controller 604 is configured to establish, monitor, and/or modify a formulation of a target fluid used during subterranean operations. The target fluid may be a fluid 637 (or variation thereof) that is tested using the testing module 670 and evaluated to be the most (or among the most) beneficial among all of the fluids 637 in terms of production of the wellbore 120. The target fluid identification module 752 may evaluate the target fluid by comparing actual results (e.g., based on applying measurements made by one or more sensor devices 660 to one or more algorithms 733) to expected results (e.g., part of the stored data 734). The target fluid identification module 752 may operate using one or more protocols 732, one or more algorithms 733, and/or stored data 734.

The communication module 707 of the controller 604 determines and implements the communication protocol (e.g., from the protocols 732 of the storage repository 731) that is used when the control engine 706 communicates with (e.g., sends signals to, obtains signals from) the user systems 655, the sensor devices 660, the network manager 680, and the other components of the testing system 600. In some cases, the communication module 707 accesses the stored data 734 to determine which communication protocol is used to communicate with another component of the testing system 600. In addition, the communication module 707 may identify and/or interpret the communication protocol of a communication obtained by the controller 604 so that the control engine 706 may interpret the communication. The communication module 707 may also provide one or more of a number of other services with respect to data sent from and obtained by the controller 604. Such services may include, but are not limited to, data packet routing information and procedures to follow in the event of data interruption.

The timer 735 of the controller 604 may track clock time, intervals of time, an amount of time, and/or any other measure of time. The timer 735 may also count the number of occurrences of an event, whether with or without respect to time. Alternatively, the control engine 706 may perform a counting function. The timer 735 may be configured to track multiple time measurements and/or count multiple occurrences concurrently. The timer 735 may track time periods based on an instruction obtained from the control engine 706, based on an instruction obtained from a user 651, based on an instruction programmed in the software for the controller 604, based on some other condition (e.g., the occurrence of an event) or from some other component, or from any combination thereof. In certain example embodiments, the timer 735 may provide a time stamp for each packet of data obtained from another component (e.g., a sensor device 660) of the testing system 600.

The power module 730 of the controller 604 obtains power from a power supply (e.g., AC mains) and manipulates (e.g., transforms, rectifies, inverts) that power to provide the manipulated power to one or more other components (e.g., the timer 735, the control engine 706) of the controller 604, where the manipulated power is of a type (e.g., alternating current, direct current) and level (e.g., 12V, 24V, 120V) that may be used by the other components of the controller 604. In some cases, the power module 730 may also provide power to one or more of the sensor devices 660.

The power module 730 may include one or more of a number of single or multiple discrete components (e.g., transistor, diode, resistor, transformer) and/or a microprocessor. The power module 730 may include a printed circuit board, upon which the microprocessor and/or one or more discrete components are positioned. In addition, or in the alternative, the power module 730 may be a source of power in itself to provide signals to the other components of the controller 604. For example, the power module 730 may be or include an energy storage device (e.g., a battery). As another example, the power module 730 may be or include a localized photovoltaic power system.

The hardware processor 721 of the controller 604 executes software, algorithms (e.g., algorithms 733), and firmware in accordance with one or more example embodiments. Specifically, the hardware processor 721 may execute software on the control engine 706 or any other portion of the controller 604, as well as software used by the users 651 (including associated user systems 655), the network manager 680, and/or other components of the testing system 600. The hardware processor 721 may be an integrated circuit, a central processing unit, a multi-core processing chip, SoC, a multi-chip module including multiple multi-core processing chips, or other hardware processor in one or more example embodiments. The hardware processor 721 may be known by other names, including but not limited to a computer processor, a microprocessor, and a multi-core processor.

In one or more example embodiments, the hardware processor 721 executes software instructions stored in memory 722. The memory 722 includes one or more cache memories, main memory, and/or any other suitable type of memory. The memory 722 may include volatile and/or non-volatile memory. The memory 722 may be discretely located within the controller 604 relative to the hardware processor 721. In certain configurations, the memory 722 may be integrated with the hardware processor 721.

In certain example embodiments, the controller 604 does not include a hardware processor 721. In such a case, the controller 604 may include, as an example, one or more field programmable gate arrays (FPGA), one or more insulated-gate bipolar transistors (IGBTs), and/or one or more integrated circuits (ICs). Using FPGAs, IGBTs, ICs, and/or other similar devices known in the art allows the controller 604 (or portions thereof) to be programmable and function according to certain logic rules and thresholds without the use of a hardware processor. Alternatively, FPGAs, IGBTs, ICs, and/or similar devices may be used in conjunction with one or more hardware processors 721.

The transceiver 724 of the controller 604 may send and/or obtain control and/or communication signals. Specifically, the transceiver 724 may be used to transfer data between the controller 604 and the users 651 (including associated user systems 655), the sensor devices 660, the network manager 680, and the other components of the testing system 600. The transceiver 724 may use wired and/or wireless technology. The transceiver 724 may be configured in such a way that the control and/or communication signals sent and/or obtained by the transceiver 724 may be obtained and/or sent by another transceiver that is part of a user system 655, a sensor device 660, the network manager 680, and/or another component of the testing system 600. The transceiver 724 may send and/or obtain any of a number of signal types, including but not limited to radio frequency signals.

When the transceiver 724 uses wireless technology, any type of wireless technology may be used by the transceiver 724 in sending and obtaining signals. Such wireless technology may include, but is not limited to, Wi-Fi, Zigbee, VLC, cellular networking, BLE, UWB, and Bluetooth. The transceiver 724 may use one or more of any number of suitable communication protocols (e.g., ISA100, HART) when sending and/or obtaining signals.

Optionally, in one or more example embodiments, the security module 728 secures interactions between the controller 604, the users 651 (including associated user systems 655), the sensor devices 660, the network manager 680, and the other components of the testing system 600. More specifically, the security module 728 authenticates communication from software based on security keys verifying the identity of the source of the communication. For example, user software may be associated with a security key enabling the software of a user system 655 to interact with the controller 604. Further, the security module 728 may restrict receipt of information, requests for information, and/or access to information.

A user 651 (including an associated user system 655), the sensor devices 660, the network manager 680, and the other components of the testing system 600 may interact with the controller 604 using the application interface 726. Specifically, the application interface 726 of the controller 604 obtains data (e.g., information, communications, instructions, updates to firmware) from and sends data (e.g., information, communications, instructions) to the user systems 655 of the users 651, the sensor devices 660, the network manager 680, and/or the other components of the testing system 600. Examples of an application interface 726 may be or include, but are not limited to, an application programming interface, a web service, a data protocol adapter, some other hardware and/or software, or any suitable combination thereof. Similarly, the user systems 655 of the users 651, the sensor devices 660, the network manager 680, and/or the other components of the testing system 600 may include an interface (similar to the application interface 726 of the controller 604) to obtain data from and send data to the controller 604 in certain example embodiments.

In addition, as discussed above with respect to a user system 655 of a user 651, one or more of the sensor devices 660, the network manager 680, and/or one or more of the other components of the testing system 600 may include a user interface. Examples of such a user interface may include, but are not limited to, a graphical user interface, a touchscreen, a keyboard, a monitor, a mouse, some other hardware, or any suitable combination thereof.

The controller 604, the users 651 (including associated user systems 655), the sensor devices 660, the network manager 680, and the other components of the testing system 600 may use their own system or share a system in certain example embodiments. Such a system may be, or contain a form of, an Internet-based or an intranet-based computer system that is capable of communicating with various software. A computer system includes any type of computing device and/or communication device, including but not limited to the controller 604. Examples of such a system may include, but are not limited to, a desktop computer with a Local Area Network (LAN), a Wide Area Network (WAN), Internet or intranet access, a laptop computer with LAN, WAN, Internet or intranet access, a smart phone, a server, a server farm, an android device (or equivalent), a tablet, smartphones, and a personal digital assistant (PDA). Such a system may correspond to a computer system as described below with regard to FIG. 8.

Further, as discussed above, such a system may have corresponding software (e.g., user system software, sensor device software, controller software). The software may execute on the same or a separate device (e.g., a server, mainframe, desktop personal computer (PC), laptop, PDA, television, cable box, satellite box, kiosk, telephone, mobile phone, or other computing devices) and may be coupled by the communication network (e.g., Internet, Intranet, Extranet, LAN, WAN, or other network communication methods) and/or communication channels, with wire and/or wireless segments according to some example embodiments. The software of one system may be a part of, or operate separately but in conjunction with, the software of another system within the testing system 600.

FIG. 8 illustrates one embodiment of a computing device 818 that implements one or more of the various techniques described herein, and which is representative, in whole or in part, of the elements described herein pursuant to certain example embodiments. For example, a controller 604 (including components thereof, such as a control engine 706, a hardware processor 720, a storage repository 731, a power module 730, and a transceiver 724) may be considered a computing device 818. Computing device 818 is one example of a computing device and is not intended to suggest any limitation as to scope of use or functionality of the computing device and/or its possible architectures. Neither should the computing device 818 be interpreted as having any dependency or requirement relating to any one or combination of components illustrated in the example computing device 818.

The computing device 818 includes one or more processors or processing units 814, one or more memory/storage components 815, one or more input/output (I/O) devices 816, and a bus 817 that allows the various components and devices to communicate with one another. The bus 817 represents one or more of any of several types of bus structures, including a memory bus or memory controller, a peripheral bus, an accelerated graphics port, and a processor or local bus using any of a variety of bus architectures. The bus 817 includes wired and/or wireless buses.

The memory/storage component 815 represents one or more computer storage media. The memory/storage component 815 includes volatile media (such as random access memory (RAM)) and/or nonvolatile media (such as read only memory (ROM), flash memory, optical disks, magnetic disks, and so forth). The memory/storage component 815 includes fixed media (e.g., RAM, ROM, a fixed hard drive, etc.) as well as removable media (e.g., a Flash memory drive, a removable hard drive, an optical disk, and so forth).

One or more I/O devices 816 allow a user 651 to enter commands and information to the computing device 818, and also allow information to be presented to the user 651 and/or other components or devices. Examples of input devices 816 include, but are not limited to, a keyboard, a cursor control device (e.g., a mouse), a microphone, a touchscreen, and a scanner. Examples of output devices include, but are not limited to, a display device (e.g., a monitor or projector), speakers, outputs to a lighting network (e.g., DMX card), a printer, and a network card.

Various techniques are described herein in the general context of software or program modules. Generally, software includes routines, programs, objects, components, data structures, and so forth that perform particular tasks or implement particular abstract data types. An implementation of these modules and techniques is stored on or transmitted across some form of computer readable media. Computer readable media is any available non-transitory medium or non-transitory media that is accessible by a computing device. By way of example, and not limitation, computer readable media includes “computer storage media”.

“Computer storage media” and “computer readable medium” include volatile and non-volatile, removable and non-removable media implemented in any method or technology for storage of information such as computer readable instructions, data structures, program modules, or other data. Computer storage media include, but are not limited to, computer recordable media such as RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which is used to store the desired information and which is accessible by a computer.

The computer device 818 is connected to a network (not shown) (e.g., a LAN, a WAN such as the Internet, cloud, or any other similar type of network) via a network interface connection (not shown) according to some example embodiments. Those skilled in the art will appreciate that many different types of computer systems exist (e.g., desktop computer, a laptop computer, a personal media device, a mobile device, such as a cell phone or personal digital assistant, or any other computing system capable of executing computer readable instructions), and the aforementioned input and output means take other forms, now known or later developed, in other example embodiments. Generally speaking, the computer device 818 includes at least the minimal processing, input, and/or output means necessary to practice one or more embodiments.

Further, those skilled in the art will appreciate that one or more elements of the aforementioned computer device 818 is located at a remote location and connected to the other elements over a network in certain example embodiments. Further, one or more embodiments is implemented on a distributed system having one or more nodes, where each portion of the implementation (e.g., an injection system 638, the testing module 670, the post-testing fluid collection system 650) is located on a different node within the distributed system. In one or more embodiments, the node corresponds to a computer system. Alternatively, the node corresponds to a processor with associated physical memory in some example embodiments. The node alternatively corresponds to a processor with shared memory and/or resources in some example embodiments.

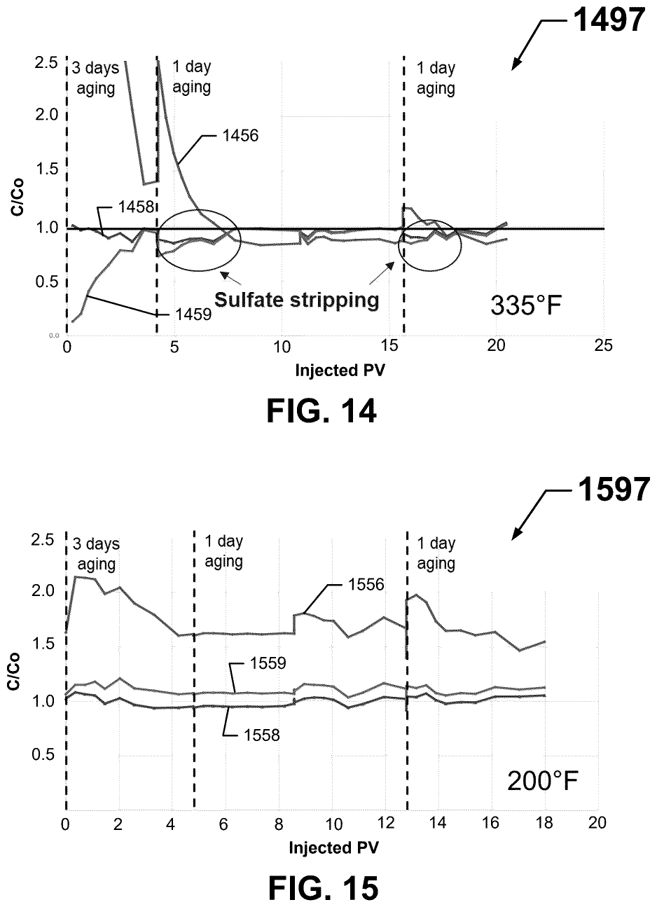

FIG. 9 shows a flowchart 999 of a method for assessing impacts of subsurface scale formation to improve hydrocarbon recovery from a fractured subterranean formation according to certain example embodiments. While the various steps in this flowchart 999 are presented sequentially, one of ordinary skill will appreciate that some or all of the steps may be executed in different orders, may be combined or omitted, and some or all of the steps may be executed in parallel. Further, in one or more of the example embodiments, one or more of the steps shown in this example method may be omitted, repeated, and/or performed in a different order.

In addition, a person of ordinary skill in the art will appreciate that additional steps not shown in FIG. 9 may be included in performing this method. Accordingly, the specific arrangement of steps should not be construed as limiting the scope. Further, a particular computing device, such as the computing device discussed above with respect to FIG. 8, may be used to perform one or more of the steps for the methods shown in FIG. 9 in certain example embodiments. Any of the functions performed below by a controller 604 may involve the use of one or more protocols 732, one or more algorithms 733, and/or stored data 734 stored in a storage repository 731.

The method shown in FIG. 9 is merely an example that may be performed by using an example system described herein. In other words, systems for evaluating a fluid for reducing scale deposition within a fractured subterranean formation may perform other functions using other methods in addition to and/or aside from those shown in FIG. 9. In certain example embodiments, the method of FIG. 9 may be performed, in full or in part, in a lab that is equipped (e.g., customized) to perform one or more of the steps discussed herein. Referring to the description above with respect to FIGS. 1A through 8, the method shown in the flowchart 999 of FIG. 9 begins at the START step and proceeds to step 981, where information about the core sample 675 inside a testing vessel 672 is obtained. As used herein, the term “obtaining” may include receiving, retrieving, accessing, generating, etc. or any other manner of obtaining the information. The testing vessel 672 may be part of a testing module 670.

The information may be obtained by a controller 604 (or an obtaining component thereof), which may include the controller 604 of FIG. 7 above, using one or more algorithms 733 and/or one or more protocols 732. The information may be obtained from a user 651, including an associated user system 655. In addition, or in the alternative, the information may be obtained from one or more sensor devices 660 that measure various parameters. Examples of the information obtained may include, but are not limited to, a composition (e.g., proppant 112, rock) of the core sample 675, size of proppant 112, rock type, size (e.g., volume) of the core sample 675, permeability, porosity, and the arrangement of the core sample 675 within the testing vessel 672 with respect to the flow of the fluid 637 (e.g., to be representative of field conditions). In certain example embodiments, the core sample 675 includes rock and proppant. The core sample 675 may be designed to be representative (e.g., in terms of permeability, in terms of porosity) of the fractured subterranean formation 110 adjacent to the wellbore 120.

The information may also additionally or alternatively be associated with the testing vessel 672 that contains the core sample 675. Information associated with the testing vessel 672 may include, but is not limited to, the dimensions (e.g., length, width, height, cross-sectional shape) of the testing vessel 672 and the material (e.g., glass, stainless steel) of the testing vessel 672. The information may be obtained at one time (e.g., prior to testing), over a period of time, periodically, or on some other basis. The information may be currently obtained data. In addition, or in the alternative, the data may be historical (e.g., data obtained from a prior field operation of the subterranean formation 110).

As an example, the core sample 675 may be measured by one or more sensor devices 660 to have a total mass (e.g., 199.31 g), a diameter (e.g., 38.10 mm), a total length (e.g., 3.42 inches), a bulk volume (e.g., 99.04 cubic cm), a porosity (e.g., 28.29%), a grain density (e.g., 2.81 g/cm³), and a pore volume (e.g., 28.02 ml). In addition, one or more calculations may be performed to determine the flow rate (e.g., 7.32 ft/day, 0.5 ml/min) that the fluid 637 is introduced into the testing vessel 672, discussed below with respect to step 982. In addition, or in the alternative, one or more calculations may be performed to determine the environmental conditions (e.g., 2300 psi confining pressure, 200 psi back pressure, temperature range of 100° F. to 330° F.) of the testing vessel 672.

In step 982, a fluid 637 is provided that flows into the testing vessel 672. Specifically, the fluid 637 flows into the testing vessel 672 and interacts with the core sample 675 within the testing vessel 672. The fluid 637 may be made up of one or more fluid components 627. Each fluid component 627 of a fluid 637 may be drawn from a fluid component source 628 using an associated injection system 638 and piping 688. The fluid 637 may be provided to flow into and/or through the testing vessel 672 using one or more injection systems 638 or an independent pumping system. The fluid components 627 of the fluid 637 may mix together naturally in a header 689 of the piping 688 and/or using a mixing module 665.

The composition of the fluid 637 may be known by a controller 604. The composition of the fluid 637 may include a specific identification/composition (e.g., CA²⁺, HCO₃⁻) of each fluid component 627 and the amount (e.g., 10 ppm, mg/L) of each fluid component 627. For example, a user 651, including an associated user system 655, may communicate the composition of the fluid 637 to the controller 604. As another example, a controller 604, using one or more protocols 732 and/or one or more algorithms 733, may determine the composition of a fluid 637 that may be tested. In such a case, a controller 604 may communicate, using one or more protocols 732, this composition to a user 651 so that the user 651 may manipulate the appropriate fluid component sources 628 and associated injection systems 638 to attain the desired fluid 637 to interact with the core sample 675. Alternatively, a controller 604 may manipulate, using one or more algorithms 733 and/or one or more protocols 732, the appropriate fluid component sources 628 and associated injection systems 638 to attain the desired fluid 637. The fluid 637 interacts with (e.g., flows through) the core sample 675 in the testing vessel 672 continually over a period of time (e.g., hours, days, months).

In certain example embodiments, a controller 604 may also set and/or control the environment to which the core sample 675 in the testing vessel 672 is exposed using one or more algorithms 733 and/or one or more protocols 732. For example, if a goal during the testing is to subject the core sample 675 in the testing vessel 672 to conditions found in the subterranean formation 110, then the controller 604 may accordingly control factors such as the temperature and the pressure (e.g., using a heater and compressor of the testing module 670) applied to the testing vessel 672. As another example, the controller 604 may control the flow rate of the fluid 637 into and/or through the testing vessel 672.

When multiple core samples 675 are tested over time, different fluids 637 (e.g., in terms of chemical composition, in terms of pH, in terms of viscosity) may be used. For example, in one test, the fluid 637 may be or include a brine with little or no scaling potential. As another example, in one test, the fluid 637 may be or include a brine that includes scaling ions (e.g., Ba and SO₄, Ca and HCO₃). As yet another example, in one test, the fluid 637 may be or include a brine that includes one or more scaling inhibitors.

In step 983, one or more parameters associated with the interaction of the fluid 637 and/or the core sample 675 in the testing vessel 672 are evaluated. Some or all of the parameters may be measured by one or more sensor devices 660. In addition, or in the alternative, some or all of the parameters may be calculated by a controller 604 using one or more algorithms 733 and/or one or more protocols 732. The measured parameters may be received from the sensor devices 660 by a controller 604. Examples of parameters that may be evaluated include, but are not limited to, a flow rate of the fluid 637 provided to the testing vessel 672, the flow rate of the post-testing fluid 657 (e.g., in the testing vessel 672, in the post-testing fluid collection system 650), the pressure of an end of the testing vessel 672 receiving the fluid 637, the pressure of an opposite end of the testing vessel 672 discharging the post-testing fluid 657, a temperature of the core sample 675 inside the testing vessel 672, an amount of sulfate in the post-testing fluid 657 and/or in the core sample 675, the permeability of the core sample 675 in the testing vessel 672, and the porosity of the core sample 675 in the testing vessel 672.

As an example, a differential pressure value (e.g., comparing the pressure before the testing vessel 672 and the pressure after the testing vessel 672) may provide information as to a change in permeability, an accumulation of scale depositions 213, and/or plugging (e.g., scale deposition 213) of the core sample 675 within the testing vessel 672. In some cases, the measured parameters may be compared to expected values or a baseline. The baseline may be established after evaluating measurements (e.g., made by one or more sensor devices 660) of parameters associated with the interaction of a fluid 637 and a core sample 675 where the fluid 637 is a brine with little or no scaling potential. In addition, or in the alternative, the measured parameters may be used as variables in one or more algorithms 733 to generate an output.

In certain example embodiments, a parameter associated with the interaction of the fluid 673 and the core sample 675 in the testing vessel 672 may be evaluated before the interaction, during the interaction, and/or after the interaction. A parameter associated with the interaction of the fluid 673 and the core sample 675 may be measured or calculated while the fluid 673 and/or the core sample 675 is inside the testing vessel 672 and/or outside the testing vessel 672. For example, one or more parameters (e.g., amount of scale deposition 213, porosity, permeability) of a core sample 675 may be measured (e.g., in the testing vessel 672, outside the testing vessel 672) using a sensor device 660 (e.g., a CT scanner, SEM/EDX, QXRD) after the interaction between a fluid 673 and the core sample 675. This information may be used to create a permeability characterization of the core sample 675.

Evaluating the parameters associated with the interaction of the fluid 673 and the core sample 675 in the testing vessel 672 may include analyzing cumulative results (e.g., across multiple tests in a stage, across tests in multiple stages of an overall testing process). For example, evaluating the parameters may include building a database to quantify the impact of scale depositions 213 on oil recovery. In such a case, a controller 604 may, for example, generate a) a plot or table of imbibed oil versus scaling potential (e.g., potential scale amount or saturation index) for a certain reaction period; b) a plot or table of change in oil recovery versus rock permeability/porosity parameters; and c) a plot or table of oil recovery results versus scale inhibitor concentration for a specific rock/brine system.

In some cases, the location, volume, and/or content of scale deposition within the core sample 675, including the fractures therein, are evaluated. Such an evaluation may be made under any circumstances (e.g., at a very low quantity relative to pore space within the core sample 675). In addition, or in the alternative, the composition of the fluid 673 and/or the post-testing fluid 657 may be assessed. In addition, or in the alternative, the interaction between in situ fluid and the core sample 675 at the approximate temperature and/or pressure at the depth range of the wellbore in the fractured subterranean formation from which the core sample 675 is obtained may be analyzed. Such analysis may include, but is not limited to, the impacts to oil and/or water mobility and/or recovery.

In step 984, a determination is made as to whether another test should be run. The determination as to whether another test should be run may be based on one or more of a number of factors, including but not limited to time, trends, actual versus predicted values, changes to a baseline, and user preferences. The determination may be made by a controller 604 using one or more algorithms 733 and/or one or more protocols 732. The determination may be based, at least in part, on information provided by a user 651, data collected from one or more sensor devices 660, results of one or more algorithms 733, and/or stored data 734 in the storage repository 731. If another test should be run, then the process proceeds to step 986. If another test should not be run, then the process proceeds to step 987.

In step 986, the testing vessel 672 is cleared. In other words, the testing vessel 672 is prepared for the next test to be conducted. The testing vessel 672 may be cleared by or using a controller 604 (or a clearing component thereof) using one or more algorithms 733 and/or one or more protocols 732. The controller 604 may also determine precisely how the testing vessel 672 should be cleaned. Alternatively, the testing vessel 672 may be cleared by a user 651. In such a case, a controller 604 may provide instructions to the user 651 (or an associated user system 655) as to how and/or whether the testing vessel 672 should be cleared. When the testing vessel 672 is cleared, the process reverts to step 981.

In step 987, a recommendation about the fluid 637 is provided. The recommendation may be provided by a controller 604 (or the target fluid identification module 752 thereof) using one or more protocols 732. The recommendation may be provided to one or more users 651 (including associated user systems 655) and/or the network manager 680. In one embodiment, a visual representation of the recommendation may be provided to one or more users 651 via an I/O device 816 such as a display, a screen, etc. The recommendation about the fluid 637 may provide any level of detail about the fluid 637, including but not limited to the precise composition of the fluid 637, the amount of time that the fluid 637 is deemed to be effective, the rate at which the fluid 637 is introduced in the wellbore 120, and the expected results (e.g., prevents accumulation of scale depositions 213, slows the accumulation of scale depositions 213 by 75%) of using the fluid 637. In certain example embodiments, the recommendation may be based on some of the other evaluations performed in step 983. When step 987 is complete, the process proceeds to the END step.

As discussed above, according to certain example embodiments, the method shown in FIG. 9 may be repeated multiple times, where each interaction of a core sample 675 with a fluid 637 may vary in terms of, for example, the composition of the fluid 637, the temperature of the testing vessel 672, and the amount of time of the interaction. For example, a testing procedure may begin with fluid 637 in the form of a brine saturation, where a saturation brine (e.g., HCl) and low sulfate sea water (LSSW) (e.g., 180 mg/L) is purged from the core sample 675 using a mixture of N₂ and CO₂ (and/or some other chemical) for a period of time (e.g., overnight). Afterwards, the core sample 675 may be vacuum saturated by NaCl, after which one or more of the properties (e.g., diameter, length, pore volume, porosity) of the core sample 675 may be measured at some temperature (e.g., 70° F.). In addition, or in the alternative, the volume of NaCl may be measured, and the saturation brine may be collected for analysis.

Continuing with the brine saturation portion of the example test, one or more core samples 675 may be saturated with a volume (e.g., 20 times the pore volume (PV) of the core sample 675) of a fluid 637 in the form of NaCl for some amount of time (e.g., 12 hours, 24 hours, a week) and measure permeability at some temperature (e.g., 70° F.). In some cases, the effluent (the post-testing fluid 657) may be collected, the volume recorded, and/or a sample analyzed. Subsequently, in some cases, the temperature of the core sample 675 within the testing vessel 672 may be increased (e.g., to 335° F.) and/or the core sample 675 may be shut in within the testing vessel 672 for some period of time (e.g., overnight, 24 hours, a week).

The next step in the example testing procedure may include a cycle in a round of a particular temperature (e.g., 335° F.). For instance, a volume (e.g., 3 times the PV of the core sample 675) of a fluid 637 in the form of a LSSW with some amount of a tracer may be injected at a flow rate (e.g., 0.5 ml/min) through the core sample 675 in the testing vessel 672 for some period of time (e.g., 12 hours, 24 hours, a week). A portion (e.g., 10 ml) of the injection brine (the fluid 637) and/or the post-testing fluid 657 may be collected and analyzed to establish a baseline. In some cases, the flow rate of the fluid 637 may be changed (e.g., to 1.0 ml/min, 2.0 ml/min) and/or maintained for some period of time on one or more occasions. Subsequently, in some cases, the core sample 675 may be shut in (with or without the fluid 637) within the testing vessel 672 for some period of time (e.g., overnight, 24 hours, a week).

The next step in the example testing procedure may include another cycle in the round of a particular temperature (e.g., 335° F.). For instance, a volume (e.g., 3 times the PV of the core sample 675) of another fluid 637 in the form of a LSSW without any tracer may be injected at a flow rate (e.g., 0.5 ml/min) through the core sample 675 in the testing vessel 672 for some period of time (e.g., 12 hours, 24 hours, a week). A portion (e.g., 10 ml) of the injection brine (the fluid 637) and/or the post-testing fluid 657 may be collected and analyzed to establish another baseline. In some cases, the flow rate of the fluid 637 may be changed (e.g., to 1.0 ml/min, 2.0 ml/min) and/or maintained for some period of time on one or more occasions. Subsequently, in some cases, the core sample 675 may be shut in (with or without the fluid 637) within the testing vessel 672 for some period of time (e.g., overnight, 24 hours, a week).

The next step in the example testing procedure may include yet another round of a particular temperature (e.g., 335° F.). For instance, a volume (e.g., 3 times the PV of the core sample 675) of another fluid 637 in the form of a LSSW with some amount of a tracer may be injected at a flow rate (e.g., 0.5 ml/min) through the core sample 675 in the testing vessel 672 for some period of time (e.g., 12 hours, 24 hours, a week). A portion (e.g., 10 ml) of the injection brine (the fluid 637) and/or the post-testing fluid 657 may be collected and analyzed to establish yet another baseline. In some cases, the flow rate of the fluid 637 may be changed (e.g., to 1.0 ml/min, 2.0 ml/min) and/or maintained for some period of time on one or more occasions. Subsequently, in some cases, the core sample 675 may be shut in (with or without the fluid 637) within the testing vessel 672 for some period of time (e.g., overnight, 24 hours, a week). When the temperature of the current step is substantially the same as the temperature of the preceding step, the current and preceding steps may be part of a series sometimes called a temperature sweep. A temperature sweep may have any of a number (e.g., 3, 4, 8, 14, 17, 22) of steps.

The next step in the example testing procedure may include still another round of a particular temperature (e.g., 335° F.). For instance, a volume (e.g., 3 times the PV of the core sample 675) of another fluid 637 in the form of a LSSW without any tracer may be injected at a flow rate (e.g., 0.5 ml/min) through the core sample 675 in the testing vessel 672 for some period of time (e.g., 12 hours, 24 hours, a week). A portion (e.g., 10 ml) of the injection brine (the fluid 637) and/or the post-testing fluid 657 may be collected and analyzed to establish still another baseline. In some cases, the flow rate of the fluid 637 may be changed (e.g., to 1.0 ml/min, 2.0 ml/min) and/or maintained for some period of time on one or more occasions. Subsequently, in some cases, the core sample 675 may be shut in (with or without the fluid 637) within the testing vessel 672 for some period of time (e.g., overnight, 24 hours, a week). In addition, or in the alternative, the temperature of the core sample 675 within the testing vessel 672 may be changed (e.g., reduced to 200° F., reduced to 100° F.) to start a new temperature sweep.

The next step in the example testing procedure may include collecting one or more effluent samples for analysis. For example, a sample (e.g., 1 ml) may be collected from the fluid 637 and/or the post-testing fluid 657 to test for SO₄ using one or more sensor devices 660 (e.g., in the form of a spectrometer). As another example, a sample (e.g., 5 ml) may be collected from the fluid 637 and/or the post-testing fluid 657 to test for Cl, SO₄, Ba, Ca, Mg, and Sr using one or more sensor devices 660.

In some cases, one or more of the preceding steps in this example testing procedure may be repeated or omitted at the particular temperature (e.g., 335° F.). For example, rather than having two cycles each at a particular temperature (e.g., 335° F.) using a fluid 637 in the form of a LSSW with some amount of a tracer and a fluid 637 in the form of a LSSW with no tracer, an alternative testing procedure may have one cycle each or more than two cycles each within a temperature sweep. In addition, or in the alternative, there may be more or fewer cycles at a particular temperature (e.g., 335° F.) using a fluid 637 in the form of a LSSW with some amount of a tracer compared to the number of cycles at the particular temperature (e.g., 335° F.) using a fluid 637 in the form of a LSSW with no tracer.

In addition, or in the alternative, one or more cycles may be performed at another particular temperature (e.g., 100° F., 200° F.) as part of another temperature sweep. For instance, after performing a temperature sweep having four cycles at one particular temperature (e.g., 350° F.), where two of the cycles (e.g., the first and the third) use a fluid 637 in the form of a LSSW with some amount of a tracer and the other two cycles (e.g., the second and the fourth) use a fluid 637 in the form of a LSSW with no tracer, another temperature sweep having four additional cycles may be performed at another particular temperature (e.g., 200° F.), where two of the cycles (e.g., the first and the third) use a fluid 637 in the form of a LSSW with some amount of a tracer and the other two cycles (e.g., the second and the fourth) use a fluid 637 in the form of a LSSW with no tracer. From there, yet another temperature sweep having another four additional cycles may be performed at another particular temperature (e.g., 100° F.), where two of the cycles (e.g., the first and the third) use a fluid 637 in the form of a LSSW with some amount of a tracer and the other two cycles (e.g., the second and the fourth) use a fluid 637 in the form of a LSSW with no tracer.

In some cases, any of the above steps and/or any additional steps in this example procedure may include a sequential injection of a fluid 637 in the form of LSSW (with or without a tracer) at a particular temperature (e.g., 100° F., 200° F., 335° F.). For instance, after a shut in period that lasts some amount of time (e.g., one day, four days, a week), a fluid 637 in the form of LSSW with some amount (e.g., 100 ppm) of a tracer may be injected into the testing vessel 672 with the core sample 675 and shut in for some period of time (e.g., overnight, 24 hours, 3 days, a week). In some cases, after the shut in period has ended, the post-testing fluid 657 may be collected and analyzed using one or more sensor devices 660.

Subsequently, a fluid 637 in the form of LSSW without any tracer may be injected into the testing vessel 672 with the core sample 675 and shut in for some period of time (e.g., overnight, 24 hours, 3 days, a week). The shut in period for this part of the sequential injection may be the same as, or different than, the shut in period for the prior part of the sequential injection. In some cases, after the shut in period has ended, the post-testing fluid 657 may be collected and analyzed using one or more sensor devices 660.

This sequential injection may be repeated any number (e.g., 2, 3, 4, 6, 10, 25) of times. In some cases, one of the two steps of the sequential injection listed in the prior paragraph may be skipped one or more times. In addition, or in the alternative, each of the various factors (e.g., temperature, amount of a tracer, shut in period, amount of the fluid 637 in the form of LSSW, collection and analysis (e.g., including sensor devices 660 used, including models and/or other algorithms 733 run) of the post-testing fluid 657) of one part of the sequential injection may be the same as, of different than, the corresponding factors of another part of the sequential injection.

As a subset of the above process to investigate and reduce the impact of scale depositions 213 on frac face 102 and/or rock on hydrocarbon recovery, an overall test having three stages may be designed to start with a series of imbibition tests using a fluid 637 in the form of a brine base with no scaling potential to interact with a series of core samples 675 taken from a range of depths within a wellbore 120. In this way, the porosity, permeability, and other characteristics of the core samples 675 may be substantially similar to each other across all of the tests. The results (e.g., measurements made by sensor devices 660, results of algorithms 733) of this initial series of imbibition tests may be used as a baseline.

The second stage of this example overall test may be designed to include a series of imbibition tests using a fluid 637 in the form of a brine base that includes (e.g., naturally occurring in the fluid 637, as an additive to the fluid 637) scaling ions (e.g., Ba and SO₄, Ca and HCO₃) to interact with a series of core samples 675 taken from the same range of depths within the wellbore 120. The results (e.g., measurements made by sensor devices 660, results of algorithms 733) of this second series of imbibition tests may be collected for comparison with the baseline established in the first series of tests and/or with the results of the third stage of the example overall test.

The third stage of this example overall test may be designed to include a series of imbibition tests using a fluid 637 in the form of a brine base that includes (e.g., naturally occurring in the fluid 637, as an additive to the fluid 637) a scale inhibitor to interact with a series of core samples 675 taken from the same range of depths within the wellbore 120. The results (e.g., measurements made by sensor devices 660, results of algorithms 733) of this third series of imbibition tests may be collected for comparison with the baseline established in the first series of tests and/or with the results of the second stage of the example overall test.

The fluid 637 used in any stage and/or in one or more tests within a stage may include other chemicals, including but not limited to a surfactant. In some cases, other types of rock samples (e.g., end trim, cuttings) from the same range of depths within the wellbore 120 may be included with a core sample 675 or used instead of a core sample 675 in any stage and/or in one or more tests within a stage. In some cases, the fluid 637 that interacts with a core sample 675 may be stirred (e.g., continuously, intermittently, at a constant rate, at a variable rate) and/or otherwise agitated for all tests within a stage or one or more tests within a stage.

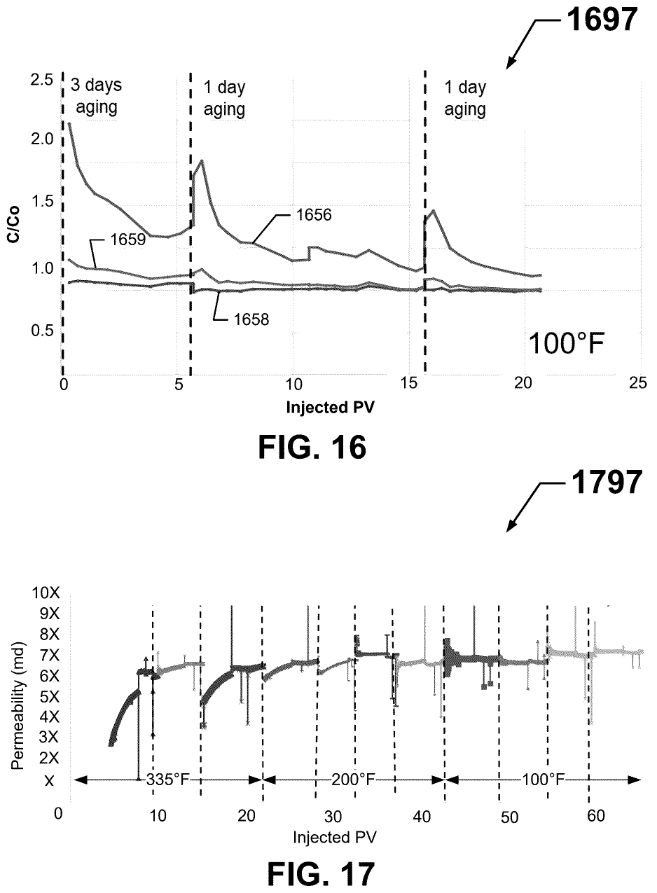

According to example embodiments, a study was conducted to perform a comprehensive coreflood investigation of sulfate release and stripping during injections of HSSW and LSSW using reservoir core samples 675. The study and its results are captured in SPE 231784 entitled “The Investigation of Sulfate Stripping Effects on Sea Water Injection in Deepwater Operation”, which is hereby incorporated by reference in its entirety and is expected to publish in May 2026. High-temperature experiments (e.g., up to 335° F.) were conducted to quantify sulfate release from the reservoir core samples 675, characterize the effects of water-rock interactions on effluent chemistry, and assess the effectiveness of sulfate stripping as a natural scale mitigation mechanism. By conducting tests on the reservoir core samples 675 according to example embodiments, insights for designing advanced scale management approaches in deepwater reservoirs may be gained. For example, conducting tests on the reservoir core samples 675 according to example embodiments may allow for quantifying sulfate release from core plugs during HSSW and LSSW injection, evaluating the temperature dependence and kinetics of sulfate stripping, and assessing implications for field scale management strategies. FIGS. 10 through 18 show graphs based on results of a series of experiments conducted on core samples 675 according to certain example embodiments. The following description of the experiments using example embodiments, including FIGS. 10 through 18, refers to the description above with respect to FIGS. 1A through 9.

Composite core samples 675 and a testing module 670 rated to 3,750 psi and 400° F. according to certain example embodiments are used in the coreflood experiments. It is noted that composite core samples 675 may have limitations in representing the entire reservoir stripping process. In the reservoir, the injected seawater reacts with larger rock and formation water volume, and the stripping effect may be enhanced. The tests in this experiment were carried out under reservoir-representative temperature ranges of 100° F. (near injector) to 335° F. (away from injector). Multiple injection tests of high-sulfate seawater (HSSW) and low-sulfate seawater (LSSW) were run, incorporating aging periods (hours to several days) and temperature conditions to simulate subsurface conditions. Initially, each core sample 675 was saturated with formation brine at approximately 70° F. During testing, there was a sequential injection of low sulfate sea water (LSSW (e.g., 180 mg/L SO₄)) and high sulfate sea water (HSSW (e.g., 2775 mg/L SO₄)) at various temperatures (in this case, 100-335° F.). The ions monitored during testing are shown in Table 1 below, and permeability of each core sample 675 is monitored throughout testing. The composition of each brine used in the coreflood experiments is presented in Table 1.

TABLE 1

| Ion | Saturation brine | LSSW | HSSW |
|---|---|---|---|
| Na⁺ (mg/L) | 102,618 | 9,983 | 11,376 |
| K⁺ (mg/L) | 11,046 | 351 | 351 |
| Mg²⁺ (mg/L) | 558 | 318 | 318 |
| Ca²⁺ (mg/L) | 3,000 | 227 | 227 |
| Cl⁻ (mg/L) | 175,000 | 16,606 | 16,411 |
| SO₄²⁻ (mg/L) | 180 | 180 | 2,775 |
| Bicarbonate (mg/L) | 0 | 0 | 0 |
| CH3COO⁻ (mg/L) | 0 | 503 | 503 |
| CO₂ (%) | 0 | 100 | 100 |
| Calculated pH under | 6.27 | 4.9 | 4.9 |
| STP | | | |

In this testing regimen using example embodiments, effluent samples were analyzed using Inductively Coupled Plasma (ICP) and Ion Chromatography (IC) to monitor ion concentrations including sulfate (S) and calcium (Ca). Permeability changes were tracked for each test to evaluate mineral dissolution or precipitation effects. Twenty-five core samples 675 from downdip reservoir intervals were screened based on X-ray fluorescence (XRF) counts for Ca and S, as well as porosity and permeability. Fourteen of these core samples 675 were categorized into low, average, and high Ca and S concentrations groups, as shown in Table 2 below. For the initial testing using the example testing module 670, core samples 675 with average Ca and S were used to reduce compositional bias. Composite stacks were prepared as follows: Core samples 675 #1 and #2 were stacked together for LSSW tests, while core samples 675 #3 and #4 were similarly stacked for HSSW testing.

TABLE 2

| | Composite | | | |
|---|---|---|---|---|
| Sample # | Stack | S (count) | Ca (count) | Fe (count) |
| 1 | 1 | < LOD (limit of | 2,912 | 16,139 |
| | | detection) | | |
| 2 | 1 | 1659 | 1,035 | 17,269 |
| 3 | 2 | 12842 | 12,373 | 28,828 |
| 4 | 2 | 8262 | 9,095 | 24,450 |

Performing a coreflood test on a core sample 675 using LSSW may be used to simulate sea water treated by SRU. These coreflood tests may be used to evaluate whether in-situ sulfate release from water-rock interactions could offset the benefit of topsides sulfate removal. These tests were conducted to investigate the effectiveness of LSSW injection under commingling of formation water and injection water at both the inlet (injection) and the outlet (production) of the testing vessel 672. When there is a significant release of SO₄ from rock to water under LSSW injection, SRU may not be a cost-effective or a desired approach in reducing subsurface sulfate scaling risk under field conditions.

FIG. 10 shows one result of this testing. Specifically, FIG. 10 shows a graph 1097 of sulfate content from testing multiple core samples 675 over time according to certain example embodiments. More specifically, the graph 1097 of FIG. 10 shows sulfate over a cumulative amount of injected PV of fluid 637 when one or more fluids 637 interacts with multiple core samples 675 in a testing vessel 672 of the testing module 670 according to the method discussed above. The graph 1097 of FIG. 10 shows 12 different baseline levels 1092 of sulfate before injection of a fluid 637 into a testing vessel 672 to interact with a core sample 675. In other words, there are 12 cycles shown in the graph 1097 of FIG. 10.

The plot 1093 in the graph 1097 represents measurements of sulfate in the post-testing fluid 657 after the fluid 637 interacts with a core sample 675. The graph 1097 also shows eight (8) aging periods 1096 (aging period 1096-1, aging period 1096-2, aging period 1096-3, aging period 1096-5, aging period 1096-6, aging period 1096-7, and aging period 1096-8) that represent a one day aging period. Further, the graph 1097 shows three non-shut in periods 1094 (non-shut in period 1094-1, non-shut in period 1094-2, and non-shut in period 1094-3) where the fluid 637 is not shut in with the core sample 675 in the testing vessel 672. Finally, the graph 1097 shows three different temperatures of testing. Specifically, the first four cycles are tested at a temperature of 335° F., the next four cycles are tested at a temperature of 200° F., and the final four cycles are tested at a temperature of 100° F.

The graph 1097 shows that the sulfate concentration in the fluid 637 (e.g., LSSW) is higher at lower temperatures. The graph 1097 also shows that the sulfate concentration is positively correlated with reaction time. Also, testing the post-testing fluid 657 may reveal an amount of sulfate (e.g., 1.14 mg of sulfate per ml) released from the core samples 675 during brine saturation and temperature sweep steps based on mass balance. In summary, FIG. 10 shows that with 1-day aging steps at 335° F., 200° F., and 100° F., SO₄ in the effluent increased after each shut-in (i.e., each aging period 1096), with larger spikes at lower temperatures. SO₄ concentrations after 1-day shut-in (aging period 1096) were in the range of 200-250 mg/L at 335° F., 300-400 mg/L at 200° F., and 400-500 mg/L at 100° F.). The purpose of the one-day shut-ins (the aging periods 1096) is to induce a measurable SO₄ concentration spike for monitoring subsequent changes. The non-shut in periods 1094 depicted in FIG. 10 represent results from overnight injection without a shut-in period, where no SO₄ concentration spike was observed.

FIG. 11 shows a graph 1197 that compares normalized Ca, Mg, and SO₄ changes during the core flood testing. Normalized concentrations (C/C.) show persistent SO₄ elevation at temperature of 200-100° F. (as against higher temperatures of 335° F.) relative to inflow LSSW, accompanied by elevated Ca. This is likely due to dissolution of Ca and SO₄ bearing minerals (e.g., anhydrite). The graph 1197 shows that normalized Ca, Mg, and SO₄ levels change during LSSW injection at three temperatures. The high initial Ca concentration in the effluent is due to high Ca concentration in the saturation brine, which is about 13 times higher than that of LSSW. There are higher SO₄ concentrations in LSSW in effluent throughout the test, especially at temperatures of 200° F. and 100° F. There are only minor changes in Mg, especially at temperatures of 200° F. and 100° F. For the normalized concentrations represented on the vertical axis, C represents the measured concentrations of ions, and Co represents initial injection.

FIG. 12 shows a graph 1297 of changes in permeability during the testing of multiple core samples 675 according to certain example embodiments. Specifically, the graph 1297 of FIG. 12 shows permeability over a cumulative amount of injected PV of fluid 637 when a fluid 637 in the form of LSSW interacts with multiple core samples 675 in a testing vessel 672 of the testing module 670 at a temperature of 100° F. according to the method discussed above. The graph 1297 of FIG. 12 shows a plot 1293 of permeability for 8 different cycles of testing.

In the first cycle, plot 1293-1 is based on the interaction of a fluid 637 that includes a tracer with a core sample 675 in a testing vessel 672. The plot 1293-1 includes an aging point 1296-1 that lasts 4 days. In the second cycle, plot 1293-2 is based on the interaction of a fluid 637 that has no tracer with another core sample 675 in a testing vessel 672. The plot 1293-2 includes an aging point 1296-2 that lasts 1 day. In the third cycle, plot 1293-3 is based on the interaction of a fluid 637 that includes a tracer (e.g., the same tracer used in the first cycle) with yet another core sample 675 in a testing vessel 672. The plot 1293-3 includes an aging point 1296-3 that lasts 1 day.

In the fourth cycle, plot 1293-4 is based on the interaction of a fluid 637 that has no tracer with another core sample 675 in a testing vessel 672. The plot 1293-4 includes an aging point 1296-4 that lasts 1 day. In the fifth cycle, plot 1293-5 is based on the interaction of a fluid 637 that includes a tracer (e.g., the same tracer used in the first cycle) with yet another core sample 675 in a testing vessel 672. The plot 1293-5 includes an aging point 1296-5 that lasts 3 days. In the sixth cycle, plot 1293-6 is based on the interaction of a fluid 637 that has no tracer with another core sample 675 in a testing vessel 672. The plot 1293-6 includes an aging point 1296-6 that lasts 1 day.

In the seventh cycle, plot 1293-7 is based on the interaction of a fluid 637 that includes a tracer (e.g., the same tracer used in the first cycle) with yet another core sample 675 in a testing vessel 672. The plot 1293-7 includes an aging point 1296-7 that lasts 1 day. In the eighth cycle, plot 1293-8 is based on the interaction of a fluid 637 that has no tracer with another core sample 675 in a testing vessel 672. The plot 1293-8 includes an aging point 1296-8 that lasts 1 day. The graph 1297 shows that there is an increase (e.g., by 11.4%) in permeability in the eighth cycle compared to the first cycle. The graph 1297 also shows that the continued, progressive increase in permeability during the sequence LSSW injection stages provides evidence that dissolution continues to occur through rock-LSSW interaction. This observation is consistent with dissolution behavior of Ca and SO₄ bearing minerals (e.g., anhydrite), which has higher solubility at lower temperature. The continued increase in permeability during the prolonged LSSW injection cycles provides evidence that dissolution continues to occur through LSSW-rock interactions. In this experiment, injection of a fluid 637 in the form of LSSW led to a 59.2% increase in permeability at lower temperatures (100° F.-200° F.), indicating anhydrite dissolution.

FIG. 13 shows another result of this testing. Specifically, FIG. 13 shows another graph 1397 of sulfate content from testing multiple core samples 675 over time according to certain example embodiments. More specifically, the graph 1397 of FIG. 13 shows sulfate over a cumulative amount of injected PV of fluid 637 when a fluid 637 in the form of LSSW interacts with multiple core samples 675 in a testing vessel 672 of the testing module 670 at a temperature of 100° F. according to the method discussed above. The graph 1397 of FIG. 13 shows 8 different baseline levels 1392 of sulfate before injection of a fluid 637 into a testing vessel 672 to interact with a core sample 675. In other words, there are 8 cycles shown in the graph 1397 of FIG. 13.

The plot 1393 in the graph 1397 represents measurements of sulfate in the post-testing fluid 657 after the fluid 637 interacts with a core sample 675. The graph 1397 also shows 8 aging periods 1396 (aging period 1396-1, aging period 1396-2, aging period 1396-3, aging period 1396-4, aging period 1396-5, aging period 1396-6, aging period 1396-7, and aging period 1396-8). In this case, aging period 1396-1 represents a four day period, aging period 1396-5 represents a three day period, and each of the remaining six aging periods 1396 (aging period 1396-2, aging period 1396-3, aging period 1396-4, aging period 1396-6, aging period 1396-7, and aging period 1396-8) represent a one day aging period. The graph 1397 shows that sulfate continues to be released from the core samples 675 even after the temperature sweep stages (as shown in FIG. 10 above). Also, testing the post-testing fluid 657 may reveal an amount of sulfate (e.g., 0.98 mg of sulfate per ml) released from the core samples 675 during sequential injection of the fluid 637 in the form of LSSW at 100° F.

After completing the temperature sweep, LSSW was injected at a constant temperature of 100° F. FIG. 13 illustrates the changes in SO₄ levels with varying shut-in durations (the aging periods 1396). The testing results show that sulfate is being released from the rock even after the temperature sweep stages (~30 PV). The testing results also show that SO₄ concentration increase is positively associated with reaction time or shut-in time. In this experiment, continued injection of a fluid 637 in the form of LSSW at 100° F. with aging steps led to an additional 11.4% permeability increase and further sulfate release, confirming ongoing mineral dissolution.

The total sulfate released from the core samples 675 may provide information about an amount of scale depositions 213 to achieve in order to maximize (or at least increase) the production of subterranean resources 111 (e.g., hydrocarbons) from a subterranean formation 110. Table 3 below shows an example of measurements that may be taken by one or more sensor devices 660 with respect to SO₄ released from each interaction between a fluid 637 in the form of LSSW and a number of core samples 675. Specifically, in this experiment, integrated effluent masses yield 210.55 mg total SO₄ released over the entire LSSW program, which may be partially broken down as 92.11 mg during the three-temperature sweep and 97.45 mg during the 100° F. sequence with aging. Based on the mass balance, this was calculated as 0.57 mg of sulfate per g of rock during brine saturation and three temperature sweep steps, and 0.49 mg of sulfate per g of rock during sequential LSSW injection at 100° F. The total SO₄ released during the experiment is 1.06 mg of sulfate per g of rock.

TABLE 3

| | Released |
|---|---|
| Parameter | Sulfate (mg) |
| Brine saturation at 70° F. | 9.75 |
| Coreflood brine saturation at 70° F. | 8.86 |
| Coreflood brine saturation at 335° F. | 2.38 |
| Coreflood during temperature sweep stage | 92.11 |
| Coreflood during sequential LSSW injection at 100° F. | 97.45 |
| Total sulfate released from core sample | 210.55 |
| Total sulfate (mg of sulfate per g of rock) | 1.06 |
| Total sulfate (mg of sulfate per ml of bulk volume) | 2.13 |

While FIGS. 10 through 13 show graphs that reflect the results of using fluids 637 in the form of LSSW during the experiment to interact with core samples 675 in a testing vessel 672, FIGS. 14 through 18 show graphs that reflect the results of using fluids 637 in the form of HSSW during the experiment to interact with core samples 675 in a testing vessel 672 using example embodiments, Tests using fluids 637 in the form of HSSW for core floods investigated sulfate stripping (i.e., a net SO₄ reduction from the injected seawater due to in situ precipitation) and the competing effects of mineral dissolution/precipitation at different temperatures. These tests were conducted to investigate the effectiveness of HSSW injection under the scenario of formation water and injection water mixing in the water leg. If stripping is observed, leading to reduced sulfate concentration at the production well, then HSSW injection (without SRU installation) may be a viable solution to control and manage the risk of sulfate formation.

FIG. 14 shows a graph 1497 that plots normalized concentrations against the PV of a core sample 675 flooded by a fluid 637 in the form of HSSW in a testing chamber 672. Specifically, the graph 1497 of FIG. 14 has a plot 1456 of normalized Ca, a plot 1458 of normalized Mg, and a plot 1459 of normalized SO₄. The stripping effect shown by the graph 1497 of FIG. 14 was observed at 335° F. The plot 1459 in the graph 1497 shows that there is limited sulfate stripping with SO₄ decreasing by approximately 20% within 2 PV after the first aging and by approximately 13% within 2 PV after the second aging.

FIG. 15 shows a graph 1597 that plots normalized concentrations against the PV of a core sample 675 flooded by a fluid 637 in the form of HSSW in a testing chamber 672 at a temperature of 200° F. Specifically, the graph 1597 of FIG. 15 has a plot 1556 of normalized Ca, a plot 1558 of normalized Mg, and a plot 1559 of normalized SO₄. Higher concentrations of Ca (plot 1556) and SO₄ (plot 1559) were observed in the effluent in this case (with the temperature at 200° F.) compared to when testing was performed at 335° F., as shown in FIG. 14. This is likely due to the dissolution of Ca and SO₄-bearing minerals, such as anhydrite. This phenomenon also correlates with the increase in permeability during the experiment and aligns with the fact that anhydrite has higher solubility at lower temperatures.

FIG. 16 shows a graph 1697 that plots normalized concentrations against the PV of a core sample 675 flooded by a fluid 637 in the form of HSSW in a testing chamber 672 at a temperature of 100° F. Specifically, the graph 1697 of FIG. 16 has a plot 1656 of normalized Ca, a plot 1658 of normalized Mg, and a plot 1659 of normalized SO₄. Higher concentrations of Ca (plot 1656) and SO₄ (plot 1659) were observed in the effluent in this case (with the temperature at 100° F.) compared to when testing was performed at 335° F., as shown in FIG. 14. This is likely due to the dissolution of Ca and SO₄-bearing minerals, such as anhydrite. This phenomenon also correlates with the increase in permeability during the experiment and aligns with the fact that anhydrite has higher solubility at lower temperatures.

FIG. 17 shows a graph 1797 of changes in permeability during the testing of multiple core samples 675 according to certain example embodiments. Specifically, the graph 1797 of FIG. 17 shows permeability over a cumulative amount of injected PV of fluid 637 when a fluid 637 in the form of HSSW interacts with a core sample 675 in a testing vessel 672 of the testing module 670 at various temperatures (specifically, 335° F., 200° F., and 100° F.) according to the method discussed herein. The graph 1797 of FIG. 17 plots permeability versus injected PV for 10 different flushing cycles of testing. Each testing cycle is separated by a one day shut-in period, represented by a vertical dashed line in the graph 1797.

In the first three cycles of flushing the fluid 637 in the form of HSSW, the temperature of the testing vessel 672 is 335° F. In the next four cycles, the temperature of the testing vessel 672 is 200° F. In the final four cycles, the temperature of the testing vessel 672 is 100° F. The initial brine permeability was 720 mD. While the testing vessel 672 is maintained at 335° F., a decrease in permeability is observed during early post-aging flushes and is attributed to likely sulfate scale deposition. At the lower temperatures of 200° F. and 100° F., an increase in permeability is noted, which is attributed to the dissolution of calcium and sulfate-bearing minerals, as confirmed by effluent analysis.

The results of the coreflood tests conducted in this experiment according to example embodiments indicate that sulfate stripping is primarily driven by water-rock interactions, where calcium ions released from carbonate minerals react with sulfate ions to form calcium sulfate. Overall, the impact of sulfate stripping was minimal, likely due to low dolomite content (~1% CaMg (CO₃)₂), which restricts dolomite dissolution even at elevated temperatures (e.g., 335° F.).

As part of the experiment, scale modeling was conducted with the effluent water ion levels from ICP analysis. The purpose of the scale modeling is to investigate the dissolution and precipitation of sulfate scales from the interactions of injected sea water with core rocks. The saturation index (SI) is a key parameter calculated by an algorithm 733 in the form of a model and is defined as the logarithm of the ratio between scaling ion activity product and the thermodynamic solubility product of a concerned scale. The SI is a measure of the degree of super saturation of the scaling ions in an aqueous system and an indication of the driving force for the supersaturated scaling ions to form scale. The higher the SI, the higher the scaling tendency. If the SI<0, the concerned scaling ions are under-saturated (no scale formation). If the SI=0, the scaling ions are at the saturation/equilibrium level. If the SI>0, the scaling ions are supersaturated (tend to form scale).

FIG. 18 shows a graph 1897 that plots SI versus effluent ion concentrations for different temperatures according to certain example embodiments. Specifically, FIG. 18 shows that, at 335° F., the SI of anhydrite (CaSO₄) is greater than 0, which indicates that anhydrite has the potential to precipitate out at this temperature. This is consistent with the sulfate concentration reduction observed during the coreflood testing at 335° F., as the sulfates got precipitated in the core sample 675. However, at 200° F. and 100° F., the SI of anhydrite is less than 0. This is because anhydrite has the potential to stay dissolved at these lower temperatures and may appear as elevated sulphate levels in the effluent, indicating no SO₄ stripping. Anhydrite dissolution is observed at lower temperatures (in this case, 200° F. and 100° F.). This indicates that precipitated anhydrite can dissolve back into the HSSW at relatively lower temperatures.

The experiment that encompassed the results shown in FIGS. 10 through 18 presents quantified evidence of sulfate release from deepwater reservoir rocks under LSSW and HSSW injection, challenging the assumption that sulfate removal prior to injection is sufficient to mitigate subsurface scaling risks. As evidenced by the results of the tests in this experiment, the temperature-dependent behavior of anhydrite dissolution underscores the importance of considering reservoir thermal profiles in scale management strategies. The coreflood tests of this experiment demonstrate a significant sulfate release during LSSW injection, with a total of approximate 1.06 mg SO₄²-/g of rock released, including from an initial brine saturation stage to sequential LSSW injection at 100° F.

In addition, the coreflood tests of this experiment demonstrate that, during HSSW injection, sulfate stripping is minimal at elevated temperatures, even though composite core samples 675 only partially reflect reservoir conditions. At lower temperatures, precipitated anhydrite is observed to re-dissolve into HSSW. This behavior could pose a potential field risk if temperature heterogeneity near the wellbore leads to precipitation of scaling ions. By removing sulfate, this process can significantly lower the risk of barite scale formation at the production well, highlighting the value of natural stripping as an effective method for managing scales. These findings may drive the decision for scale management programs in deepwater, especially for SRU. Comprehensive scale management programs may integrate real-time monitoring, reservoir reaction modeling, and adaptive control measures according to certain example embodiments in order to optimize the advantages of sulfate stripping and manage emerging risks effectively.

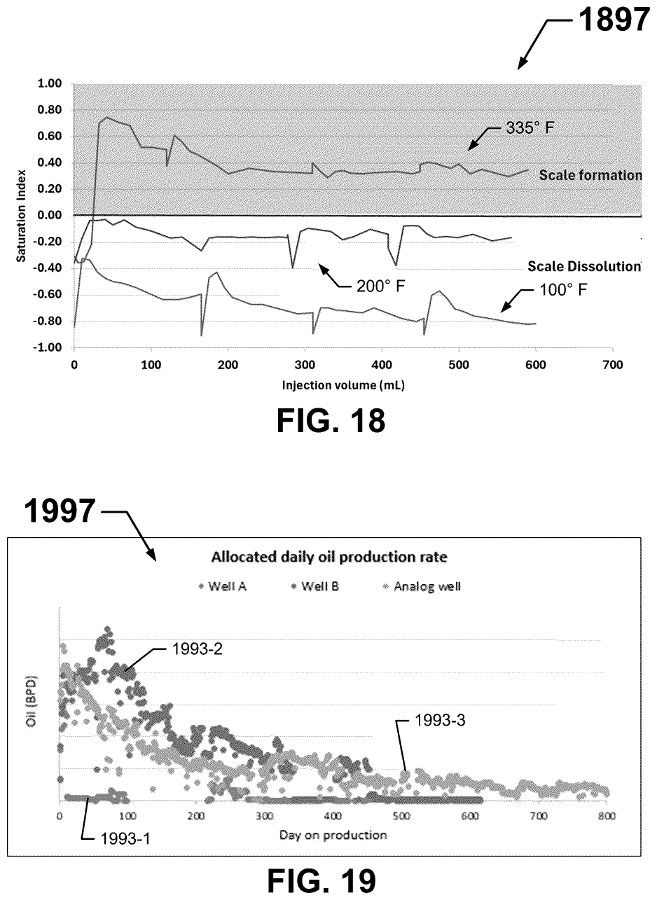

FIG. 19 shows a graph 1997 of oil production rates for multiple wellbores over time according to certain example embodiments. Referring to the description above with respect to FIGS. 1A through 18, the graph 1997 of FIG. 19 shows three plots 1993 (plot 1993-1, plot 1993-2, and plot 1993-3) of oil production (in barrels per day) over time. The three plots 1993 correspond to three wellbores (e.g., wellbore 120) that each have a horizontal section 103 that is approximately 1 mile long. The horizontal section 103 of all three wellbores 120 are drilled into the same layer of the subterranean formation 110. The horizontal section 103 of the wellbore 120 associated with plot 1993-3 is located approximately 10 miles away from the horizontal sections 103 of the wellbores 120 associated with plot 1993-1 and plot 1993-2, which are located substantially close (e.g., within a few hundred feet) of each other in the layer of the subterranean formation 110.

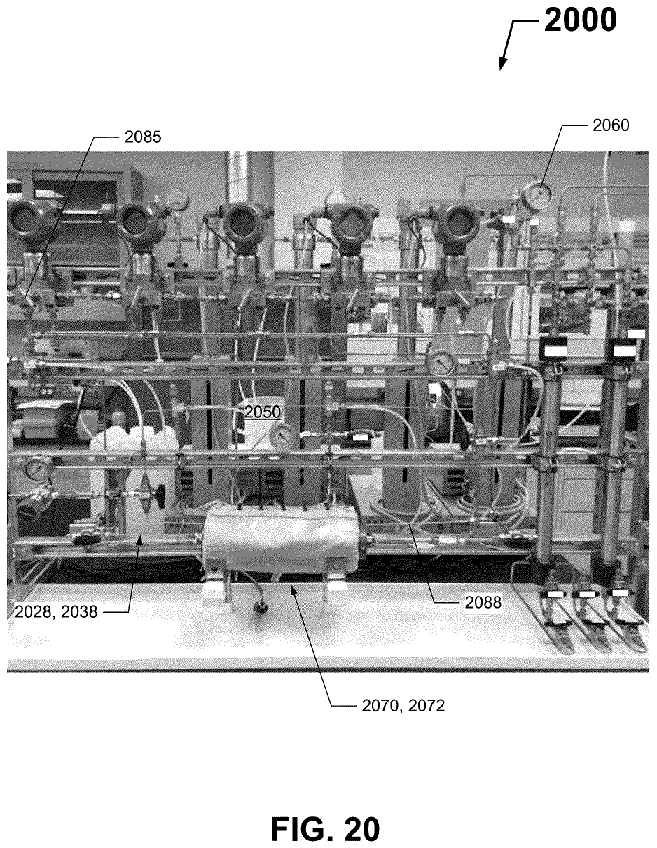

FIG. 20 shows an image of a testing system 2000 that is modeled after the testing system of FIG. 6 according to certain example embodiments. Specifically, FIG. 20 shows a front view of the testing system 2000. Referring to FIGS. 1A through 19, the testing system 2000 of FIG. 20 includes a fluid component source 2028, an injection system 2038, piping 2088, multiple sensor devices 2060, multiple valves 2085, a testing module 2070 with a testing vessel 2072 having a core sample (e.g., core sample 675, hidden from view) disposed therein, and a post-testing fluid collection system 2050. These components of the testing system 2000 are substantially similar to the corresponding components of the testing system 600 of FIG. 6.

The fluid component source 2028 of FIG. 20 provides the fluid 637 (or a component thereof). The injection system 2038 (e.g., a pump) delivers the fluid 637 to the testing vessel 2072 of the testing module 2070 through piping 2088. The flow of the fluid 637 through the piping 2088 may be controlled, at least in part, by one or more of the valves 2085. At the testing module 2070, the fluid 637 flows into and/or through the testing vessel 2072. A controller 604 of the system 2000 may control the valves 2085, the injection system 2038, and/or environmental control components (e.g., for pressure, for temperature) of the testing vessel 2072. Any of a number of sensor devices 2060 of the system 2000 measures one or more parameters associated with the interaction between the fluid 637 and the core sample 675 in the testing vessel 2072. A sensor device 2060 may measure a parameter within the testing vessel 2072, in piping 2088, and/or in the post-testing fluid collection system 2050.

In the system 2000 of FIG. 20, operating conditions may be limited (e.g., a maximum pressure of 3750 psi, a maximum pressure of 5000 psi, a maximum pressure of 7000 psi, a maximum temperature of 400° F.) based on the equipment used. These limits allow for a range of operating conditions (e.g., temperatures between 100° F. and 335° F., back pressure of 200 psi, confining pressure of 2300 psi) for testing the interaction between fluids 637 and core samples 675.

As discussed above, an example testing system (e.g., testing system 600, testing system 1400) can be used for one or more of a number of purposes using one or more of a number of analytical methods (e.g., user different sensor devices 660). Table 4 provides non-exclusive examples of some of these analytical methods and corresponding purposes.

TABLE 4

| ANALYTICAL METHOD | PURPOSE |
|---|---|
| Scanning Electron Microscope | Visualize morphology and perform elemental analysis |
| (SEM) | for materials (core sample, proppant, scales, etc.) |
| Quantitative X-ray Diffraction | Measure crystal structure and confirm scale type and |
| (QXRD) | material composition |
| Differential Pressure | Monitor pressure difference which is an indication of |
| | scale deposition on core sample inside testing vessel |
| Photograph | Overview of scale formation on a core sample |
| Inductively coupled plasma - optical | Elemental analysis for fluid samples |
| emission spectrometry (ICP-OES) | |
| Ion Chromatography (IC) | Analyze water/brine composition |
| pH probe | Measure pH in water/brine samples |
| X-ray Fluorescence (XRF) | Elemental analysis |
| Dissolution test | Solid characterization |
| X-ray mapping | Evaluate element distribution |
| Inductively coupled plasma - mass | Elemental analysis for fluid samples |
| spectrometry (ICP-MS) | |
| Particle size analyzer | Analyze particle size and distribution |
| Stable isotope analysis | Measure stable isotope ratio |

Example embodiments may be used manage multiple risks associated with developing a wellbore or series of wellbores at one time. For example, a layer of a subterranean formation under consideration for development may have high barium water invasion during fracturing and shut-in stages in adjacent wellbores prior to being put on production. In such a case, one risk to manage is determining where to place a new wellbore and creating a fracturing design may lead to identifying sources and causes of external water invasion, which may lead to an assessment of scaling risk using example embodiments. Also in such a case, another risk to manage is produced water chemistry and scale surveillance, which may lead to scale mitigation and remediation, which may lead to scale deposition at the subsurface, causing poor production output. Example embodiments may be used to manage this risk, as well.

By continuously modeling a stream of continuous information (e.g., measurements of parameters associated with interactions between fluids and core samples) related to a wellbore, example embodiments may be used to investigate the root cause and/or source of external water invasion (e.g., clarify the impact of SWD injection on external high Ba water invasion). In addition, or in the alternative, example embodiments may be used to confirm and/or improve the understanding of the root cause for production underperformance. In addition, or in the alternative, example embodiments may be used to optimize the remediation and/or restoration plans for one or more wellbores. In addition, or in the alternative, example embodiments may be used to devise life-of-well produced water/scale surveillance and management programs for future wellbores in a production field.

In some cases, example embodiments are directed to a method for assessing impacts of subsurface scale formation from fluid flow to improve hydrocarbon recovery from a fractured subterranean formation, where the method includes obtaining a plurality of first measurements, made by a sensor device, of a parameter associated with a first interaction between a first core sample and a first fluid of a plurality of fluids inside of a testing vessel, wherein the first core sample is extracted from a depth range of a wellbore in the fractured subterranean formation, and wherein the first fluid comprises a first brine without a scale inducer; establishing a baseline of the parameter using the plurality of first measurements; obtaining a plurality of second measurements, made by the sensor device, of the parameter associated with a second interaction between a second core sample and a second fluid of the plurality of fluids inside of the testing vessel, wherein the second core sample is extracted from the depth range of the wellbore in the fractured subterranean formation, and wherein the second fluid comprises a second brine and a scale impact additive; comparing the plurality of second measurements to the baseline; and identifying, based on comparing the plurality of second measurements to the baseline, a target fluid for use in production of a hydrocarbon from the fractured subterranean formation within the depth range.

In such cases, the method may also include controlling, during the first interaction, a temperature and a pressure applied to the first core sample, where the temperature and the pressure are substantially the same as found in the depth range of the wellbore. In addition, or in the alternative, in such cases, the first core sample and the second core sample may have a permeability and a porosity that are substantially similar to each other. In addition, or in the alternative, in such cases, the second fluid may include low sulfate seawater and a tracer. In addition, or in the alternative, in such cases, the second interaction may last for at least 24 hours.

In some cases, example embodiments are directed to a system for assessing impacts of subsurface scale formation from fluid flow to improve hydrocarbon recovery from a fractured subterranean formation, where the system includes a testing module comprising a testing vessel, wherein the testing vessel is configured to receive a plurality of core samples extracted from a depth range of a wellbore in the fractured subterranean formation, wherein the testing module is configured to control a pressure and a temperature of the testing vessel, wherein the testing module is configured to facilitate: a first fluid of a plurality of fluids that interacts with a first core sample of the plurality of core samples in the testing vessel for a first period of time, wherein the first fluid comprises a first brine without a scale inducer; and a second fluid of the plurality of fluids that interacts with a second core sample of the plurality of core samples in the testing vessel for a second period of time, wherein the second fluid comprises a second brine and a scaling ion. In such cases, system may also include a plurality of sensor devices configured to measure a plurality of parameters associated with an interaction between one of the plurality of core samples and one of the plurality of fluids within the testing vessel. In such cases, the plurality of parameters may include an amount of sulfate scaling on the plurality of core samples.

Example embodiments may be used for assessing impacts of subsurface scale formation to improve hydrocarbon recovery from a fractured subterranean formation. Example embodiments may be used to fully or partially automate the process of identifying and/or generating different fluids from fluid components, providing the fluid that flows to interact with a core sample in a testing vessel of a testing module, and evaluating the impact of the fluid relative to scale deposition and production of subterranean resources. Example embodiments may also communicate the results of an evaluation of a fluid, determine alternative fluids that may be more effective, generate those alternative fluids, and/or evaluate those alternative fluids during and after testing. Using example embodiments, the core samples that are tested may be subjected to conditions that are representative of those of a fractured subterranean formation. Example embodiments may provide a number of benefits. Such benefits may include, but are not limited to, ease of use, extending the life of a producing well, optimize use of proppant in fractures, flexibility, configurability, and compliance with applicable industry standards and regulations.

Although embodiments described herein are made with reference to example embodiments, it should be appreciated by those skilled in the art that various modifications are well within the scope and spirit of this disclosure. Those skilled in the art will appreciate that the example embodiments described herein are not limited to any specifically discussed application and that the embodiments described herein are illustrative and not restrictive. From the description of the example embodiments, equivalents of the elements shown therein will suggest themselves to those skilled in the art, and ways of constructing other embodiments using the present disclosure will suggest themselves to practitioners of the art. Therefore, the scope of the example embodiments is not limited herein.

What is claimed is:

1. A method for assessing impacts of subsurface scale formation from fluid flow to improve hydrocarbon recovery from a fractured subterranean formation, the method comprising:
obtaining a plurality of first measurements, made by a sensor device, of a parameter associated with a first interaction between a first core sample and a first fluid of a plurality of fluids inside of a testing vessel, wherein the first core sample is extracted from a depth range of a wellbore in the fractured subterranean formation, and wherein the first fluid comprises a first brine without a scale inducer;
establishing a baseline of the parameter using the plurality of first measurements;
obtaining a plurality of second measurements, made by the sensor device, of the parameter associated with a second interaction between a second core sample and a second fluid of the plurality of fluids inside of the testing vessel, wherein the second core sample is extracted from the depth range of the wellbore in the fractured subterranean formation, and wherein the second fluid comprises a second brine and a scale impact additive;
comparing the plurality of second measurements to the baseline; and
identifying, based on comparing the plurality of second measurements to the baseline, a target fluid for use in production of a hydrocarbon from the fractured subterranean formation within the depth range.

2. The method of claim 1, further comprising:
obtaining a plurality of third measurements, made by the sensor device, of the parameter associated with a third interaction between a third core sample and a third fluid of the plurality of fluids inside of the testing vessel, wherein the third core sample is extracted from the depth range of the wellbore in the fractured subterranean formation, and wherein the third fluid comprises a third brine and a scale inhibitor;
comparing the plurality of second measurements and the plurality of third measurements to the baseline; and
confirming, based on comparing the plurality of third measurements to the baseline, the target fluid for use in production of the hydrocarbon from the fractured subterranean formation within the depth range.

3. The method of claim 2, wherein the second interaction comprises a first coreflood test of the second core sample, and wherein the third interaction comprises a second coreflood test of the third core sample.

4. The method of claim 1, further comprising:
controlling, during the first interaction, a temperature and a pressure applied to the first core sample.

5. The method of claim 1, further comprising:
controlling, during the first interaction, a flow rate of the first fluid over the first core sample.

6. The method of claim 1, wherein the scale impact additive of the second interaction comprises a scaling ion.

7. The method of claim 1, wherein the first interaction comprises imbibition of the first fluid by the first core sample.

8. The method of claim 1, wherein the parameter comprises an amount of a sulfate ion.

9. The method of claim 1, further comprising:
evaluating a post-testing fluid after the second interaction, wherein the post-testing fluid comprises the second fluid after the second interaction.

10. The method of claim 1, wherein the parameter is associated with a location of scale deposition within the first core sample and the second core sample.

11. A system for assessing impacts of subsurface scale formation from fluid flow to improve hydrocarbon recovery from a fractured subterranean formation, the system comprising:
a testing module comprising a testing vessel, wherein the testing vessel is configured to receive a plurality of core samples extracted from a depth range of a wellbore in the fractured subterranean formation, wherein the testing module is configured to control a pressure and a temperature of the testing vessel, wherein the testing module is configured to facilitate:
a first fluid of a plurality of fluids that interacts with a first core sample of the plurality of core samples in the testing vessel for a first period of time, wherein the first fluid comprises a first brine without a scale inducer; and
a second fluid of the plurality of fluids that interacts with a second core sample of the plurality of core samples in the testing vessel for a second period of time, wherein the second fluid comprises a second brine and a scaling ion.

12. The system of claim 11, wherein the testing module is further configured to facilitate:
a third fluid of the plurality of fluids that interacts with a third core sample of the plurality of core samples in the testing vessel for a third period of time, wherein the third fluid comprises a third brine and a scale inhibitor.

13. The system of claim 11, further comprising:
a plurality of sensor devices configured to measure a plurality of parameters associated with an interaction between one of the plurality of core samples and one of the plurality of fluids within the testing vessel.

14. The system of claim 13, further comprising:
a controller communicably coupled to the plurality of sensor devices, wherein the controller is configured to evaluate measurements of the plurality of parameters made by the plurality of sensor devices.

15. The system of claim 11, further comprising:
a plurality of fluid component sources that contain a plurality of fluid components, wherein each of the plurality of fluid component sources contains a fluid component of one of the plurality of fluids; and
a plurality of injection systems, wherein each of the plurality of injection systems is configured to move each fluid component toward the testing module.

16. The system of claim 15, further comprising:
a header located between the plurality of injections systems and the testing vessel, wherein the plurality of fluid components mix together inside the header to form one of the plurality of fluids before being introduced to the testing vessel.

17. The system of claim 15, further comprising:
a mixing module located between the plurality of injections systems and the testing vessel, wherein the mixing module mixes the plurality of fluid components to form one of the fluids.

18. The system of claim 11, further comprising:
a post-testing fluid collection system that is configured to receive a post-testing fluid from the testing module, wherein the post-testing fluid comprises one of the plurality of fluids after the one of the plurality of fluids interacts with one of the plurality of core samples in the testing vessel.

19. The system of claim 11, wherein the testing vessel is removable from the testing module.

20. The system of claim 11, further comprising:
an environmental control component that controls a temperature within the testing vessel, a pressure within the testing vessel, or any combination thereof.
